(19) United States
(12) Patent Application Publication
(10) Pub. No.: US 2023/0414605 A1
(43) Pub. Date: Dec. 28, 2023

(54) Method of Treating Cancer Using FABP5 Inhibitors

(71) Applicant: Aurigene Oncology Limited, Bangalore (IN)

(72) Inventors: Muralidhara Ramachandra, Bangalore (IN); Leena Khare, Bangalore (IN); Dinesh Chikkanna, Bangalore (IN); Vijayashankar Nataraj, Bangalore (IN); Sunil Kumar Panigrahi, Kantamal (IN)

(73) Assignee: Aurigene Oncology Limited, Bangalore (IN)

(21) Appl. No.: 18/024,625

(22) PCT Filed: Sep. 3, 2021
(86) PCT No.: PCT/IB2021/058054

§ 371 Date: Mar. 3, 2023

(30) Foreign Application Priority Data
Sep. 4, 2020 (IN) 202041038258

(51) Int. Cl. A61K 31/496; A61K 31/495; A61K 31/4468; A61K 31/439; A61K 31/18; A61K 31/40; A61K 31/165; A61P 35/00

(52) U.S. Cl. CPC A61K 31/496; A61K 31/495; A61K 31/4468; A61K 31/439; A61K 31/18; A61K 31/40; A61K 31/165; A61P 35/00

(57) ABSTRACT

The present invention describes methods of treating cancer associated with a deregulated lymphocyte receptor signaling pathway by administering to a subject in need thereof a fatty acid-binding protein 5 (FABP5) inhibitor. The present invention relates to the method of treating cancers, particularly hematological cancers and solid tumors, in a subject having a deregulated lymphocyte receptor signaling pathway by administering to a subject a FABP5 inhibitor.

### RELATED APPLICATIONS

This application claims a benefit of Indian provisional application number 202041038258, filed on 4 Sep. 2020; the specification of which are hereby incorporated by reference in their entirety.

### TECHNICAL FIELD

The present disclosure relates to methods of treating cancer by administering to a subject in need thereof a FABP5 inhibitor. Particularly, the present invention relates to the method of treating lymphoid cancers in a subject having a deregulated lymphocyte receptor signaling pathway comprising administering to a subject in need thereof a FABP5 inhibitor.

### BACKGROUND OF THE INVENTION

B cells and T cells play a major role in mounting an effective adaptive immune response. These cells express specific receptors that effectively recognize antigens: the B-cell antigen receptor (BCR) and the T-cell antigen receptor (TCR), respectively. The BCR is a transmembrane complex composed of a highly variable membrane-bound immunoglobulin of either the IgM or IgD subclass in a complex with the invariant also known as Igα and Igβ (CD79a and CD79b) heterodimer (Tolar et al. Immunol Rev 232: 34-41, 2009). The BCR Immunoglobulin sequences are highly variable because the genes that encode these proteins undergo rearrangements and somatic hypermutation during B-cell development, which produces a high degree of protein diversity (≥10 [symbol] different receptors) (Schatz and Ji, Nat Rev Immunol 11: 251-263, 2011). The TCR is also characterized by highly variable antigen-binding subunits, either an αβ or a γδ dimer (Davis, Semin Immunol 16: 239-243, 2004; Krogsgaard and Davis 2005, Nat Immunol 6: 239-245). These are coupled to the invariant CD3 subunits γε, δε, and ξξ, which are essential for trafficking and stability of the γδ and αβ subunits at the plasma membrane.

B and T lymphocytes activation is the key event in the generation of efficient adaptive immune responses and is regulated by a diverse network of signal transduction pathways. This complex signalling responsible for the activation of B- and T-cells has been studied extensively. An oncogenic activation of these cells followed by several downstream aberrant signalling mechanisms is the main cause of various lymphoid malignancies such as leukemia, lymphoma, multiple myeloma and other B-cell and T-cell cancers. A growing body of evidence supports that caspase recruitment domain family member 11 (CARD11 or CARMA1)—B cell CLL/lymphoma 10 (BCL10)—MALT1 paracaspase (MALT1) [CBM] signalosome complex is a critical regulator of NF-kB pathway leading to lymphocyte activation, proliferation, survival, metabolism and deregulation in CBM components and downstream effectors can be potentially linked with diverse group of human primary immunodeficiency diseases (Henry Y. Lu et al., Frontiers in Immunology, 2018, Vol. 9, Art. 2078). So, targeting BCR or TCR signalling pathway is considered having potential therapeutic benefit for the treatment of lymphoid malignancies and immunodeficiency diseases.

Fatty acid-binding protein-5 (FABP5) or epidermal FABP belongs to a low molecular weight lipid binding protein family. FABP5 is involved in binding, storing, and transporting hydrophobic ligands to the proper cellar compartment. FABP5 is involved in the uptake and transport of long chain fatty acids (LCFAs) and plays a key role in cell signalling, gene regulation, cell growth and differentiation. Recent studies have suggested that FABP5 play important roles in regulation of gene expression associated with cell growth and differentiation. FABP5 expression level was closely related to malignancy in several types of cancers. FABP5 is upregulated in some cancers, including cholangiocarcinoma and hepatocellular carcinoma (Ohata et al., Cancer Med. 2017, May 6 (5): 1049-1061, Fujii et al. Proteomics, 5: 1411-1422, 2005, Jeong et al. Oncol Rep. 2012 October; 28 (4): 1283-92), prostatic carcinomas (Al-Jameel et al., Oncotarget. 2017, May 9; 8 (19): 31041-31056 Adamson et al., Oncogene. 2003 May 8; 22 (18):2739-49; Kawaguchi et al., Biochem J. 2016 Feb. 15; 473 (4):449-61; Morgan et al., Int J Oncol. 2008 April; 32 (4):767-75; Morgan et al., PPAR Res. 2010; 2010:234629; Myers et al., J Cancer. 2016, July 5; 7 (11):1452-64, Senga et al. Oncotarget, 2018, Vol. 9, (No. 60), pp: 31753-31770; Carbonetti et al. Sci Rep. 2019, December 12; 9 (1): 18944, Carbonetti et al. Prostate. 2020, January 80 (1): 88-98), glioma (Barbus et al., 2011, J Natl Cancer Inst 103:598-606), oral squamous cell carcinoma (Fang et al., J Oral Pathol Med (2010) 39: 342-348; Masouye et al Dermatology. 1996, 192:208-213; Watanabe et al., J. Dermatol Sci. 16 (1), 17e22. 1997), cervical cancer (Wang et al., Br. J. Cancer 110 (7), 1748e1758, 2014c; Wang et al., 2016 Nov Tumour Biol. 37 (11), 14873e14883, colorectal cancer (Kawaguchi et al., Biochem. J. 473 (4), 449e461, 2016a, FEBS Open Bio 6 (3).5463.12031.2016b; Koshiyama et al., Biomed. Chromatogr. 27 (4), 440e450 2013; Petrova et al., Clin. Biochem. 41 (14e15), 1224e1236, 2008), pancreatic cancer (Sinha et al., Electrophoresis 20 (14), 2952e2960,1999), bladder cancer (Chen et al., J. Int. Med. Res. 39 (2), 533e540, 2011), breast cancer (Kannan-Thulasiraman et al., J. Biol. Chem., 285 (25), 19106e19115, 2010; Levi et al., Cancer Res. 73 (15), 4770e4780, 2013; Liu et al., Am. J. Pathol. 178 (3), 997e1008, 2011a, Mol. Cancer 14, 129, 2015a; Powell et al., Oncotarget 6 (8), 6373e6385, 2015; Thulasiraman et al., BMC Cancer 14 (724), 2014; Zhang et al., Oncotarget 6 (34), 35830e35842, 2015), gastric (Zhao et al., Oncol Lett. 2017; 14(4): 4772), Pan et al. Cancer Cell Int. 2019, 19:69), breast cancer (Levi et al., Cancer Res. 73:4770-4780, 2013), Uveal melanoma (Xu et al., Oncol Lett. 2020; 19 (3): Esophageal (Ogawa et al., 2008, Dis Esophagus 21:288-297), renal cell carcinoma (Lv et al., Int J Oncol. 2019, April; 54 (4):1221-1232). FABP5 is highly upregulated via epigenetic mechanisms during carcinogenesis (Kawaguchi, The Biochemical journal, 473 (2016) 449-461). The functions of FABP5 in modulation of cellular signaling have been extensively studied and suggested that FABP5 is involved in EGFR, VEGFR, NFkB and PPAR pathways and play a role in pathogenesis of various solid tumors. However, the direct involvement of FABP5 in aberrant signalling events downstream BCR or TCR signalling pathway and therapeutic benefit of targeting FABP5 for the treatment of lymphoid malignancies or other associated diseases are yet to be reported.

International applications WO/2009/053715, WO/2011/163195, WO/2012/154518 WO/2015/091532, WO/2015/140055, WO/2016/087994, WO/2016/106629, WO/2018/053189, WO/2019/089512, WO/2019/149164 etc., report compounds and their derivatives capable of targeting BCR signaling such as Bruton tyrosine kinase (BTK) inhibitors, PI3K isoform-specific inhibitors and SYK inhibitors and have been shown to be effective in the treatment B cell malignancies. However, these agents are active only in those instances where BCR pathway activation is due to BCR stimulation by microbial antigens or autoantigens present in the tissue microenvironment, activating mutations within the BCR complex or signaling components upstream of the targets of interest (such as BTK, PI3Kdelta and SYK depending on the inhibitor) and ligand-independent tonic BCR signaling. They do not show clinical efficacy in cancers where BCR pathway activation is due to changes downstream such as mutations in CARD11 and TNFAIP3, and other changes.

For the above stated reasons, there is a need for compounds capable of modulating lympocyte receptor pathway for the treatment of leukemia, lymphoma, multiple myeloma and other B-cell and T-cell cancers, and also for immunodeficiency diseases.

### SUMMARY OF THE INVENTION

The present disclosure is based, in part, on methods of treating cancer, comprising contacting a cancer cell with a fatty acid-binding protein 5 (FABP5) inhibitor. The present disclosure also relates to a method of inhibiting haematological cancer cell proliferation associated with a deregulated lymphocyte receptor signaling pathway, comprising contacting the cell with a fatty acid-binding protein 5 (FABP5) inhibitor.

In one aspect, the present invention relates to methods of inhibiting cancer cell proliferation associated with a deregulated lymphocyte receptor signaling pathway, comprising contacting the cell with a compound of formula (I) or a pharmaceutically acceptable salt or a stereoisomer thereof as described below.

In another aspect, the present invention relates to methods of inhibiting cancer cell proliferation associated with a deregulated lymphocyte receptor signaling pathway, comprising contacting the cell with a compound of formula (I) or a pharmaceutically acceptable salt or a stereoisomer thereof, which are capable of suppressing and/or inhibiting FABP5 activity. For example, these compounds can be used to treat one or more diseases characterized by aberrant or undesired activity of lymphocyte receptor (e.g., B-cell receptor and T-cell receptor) signaling pathways.

In further aspect, the present invention relates to inhibiting B-cell cancer cell or T-cell cancer cell proliferation by contacting a B-cell cancer cell or T-cell cancer cell with a FABP5 inhibitor. The B cell cancer can be a non-Hodgkin's lymphoma, a Hodgkin's lymphoma, a chronic lymphocytic leukaemia (CLL) or a multiple myeloma. The T-Cell cancer can be T cell leukemia or T-cell lymphoma.

In further aspect, the invention includes inhibiting the growth of a solid tumor by contacting the tumor with a FABP5 inhibitor. The solid tumour can be a tumour of the prostate, brain, head and neck, cervix, colon, pancreas, bladder, gastric, skin, esophagus, liver, bile duct or kidney.

In yet another aspect, the present invention relates to method of treating cancer having a deregulated lymphocyte receptor signaling pathway in a subject comprising administering the subject in need thereof a therapeutically effective amount of FABP5 inhibitor.

In yet another aspect, the present invention relates to method of treating hematological cancer in a subject comprising administering the subject in need thereof a therapeutically effective amount of FABP5 inhibitor.

### BRIEF DESCRIPTIONS OF THE DRAWINGS

FIG. 1: Anti-proliferative activity of Compound 23 vs. ibrutinib (BTK inhibitor) in OCI-LY3 cell line

FIG. 2: Inhibition of cellular MALT1 activity

FIG. 3A: RelB accumulation in OCI-LY10 cells on treatment with Compound 23

FIG. 3B: Inhibition of A20 cleavage in OCI-LY10 cells on treatment with Compound 23

FIG. 4A: Impact of Compound 23 on IL-6 secretion

FIG. 4B: Impact of compound 23 on IL-10 secretion

FIG. 5A: EC₅₀ of compound 23 in NF-kB reporter assay

FIG. 5B: EC₅₀ of compound 23 in NFAT reporter assay

FIG. 6A: In-vivo tumour growth inhibition of compound 23 in human DLBCL tumor model

FIG. 6B: Inhibition of circulatory IL-10 upon treatment of compound 23 in human DLBCL tumor model

FIG. 6C: Inhibition of IL-10 in the tumor upon treatment of of compound 23 in human DLBCL tumor model

FIG. 7: Cellular thermal shift assay for FABP5 in OCI-Ly10 cells

### DETAILED DESCRIPTION OF THE INVENTION

Each embodiment is provided by way of explanation of the disclosure, and not by way of limitation of the disclosure. In fact, it will be apparent to those skilled in the art that various modification and variations can be made in the present disclosure without departing from the scope or spirit of the disclosure. For instance, features illustrated or described as part of one embodiment can be used on another embodiment to yield a still further embodiment. Thus it is intended that the present disclosure cover such modifications and variations as come within the scope of the appended claims and their equivalents. Other objects, features, and aspects of the present disclosure are disclosed in, or can be derived from, the following detailed description. It is to be understood by one of ordinary skill in the art that the present discussion is a description of exemplary embodiments only, and is not to be construed as limiting the broader aspects of the present disclosure.

The present invention provides a method of modulating a deregulated lymphocyte receptor signaling pathway in a cancer cell, comprising contacting the cell with a fatty acid-binding protein 5 (FABP5) inhibitor. In certain embodiments, the disclosure provides a method of inhibiting cancer cell proliferation associated with a deregulated lymphocyte receptor signaling pathway, comprising contacting the cell with a fatty acid-binding protein 5 (FABP5) inhibitor.

In certain embodiments, lymphocyte receptor signaling is B-cell receptor signaling (BCR) or T-cell receptor signaling (TCR). In certain embodiments, lymphocyte receptor signaling is B-cell receptor signaling (BCR).

In certain embodiments, deregulated lymphocyte receptor signaling is a deregulated B-cell receptor signaling (BCR) or a deregulated T-cell receptor signaling (TCR). In certain embodiments, deregulated lymphocyte receptor signaling is a deregulated B-cell receptor signaling (BCR).

In some embodiments, the deregulated lymphocyte receptor signaling is associated with the genetic alterations in lymphocyte receptor signaling mediator. In some embodiments, the deregulated lymphocyte receptor signaling is associated with the genetic alterations in B-cell receptor signaling mediator or T-cell receptor signaling mediator.

In certain embodiments, the genetic alterations in lymphocyte receptor signaling mediator comprises mutations, deletions or other changes leading overexpression of lymphocyte receptor signaling mediator leading to over activation of lymphocytes. In certain embodiments, the genetic alterations in lymphocyte receptor signaling mediator comprises a mutation (loss of function or a deleterious or activating), a translocation, an amplification, or a genomic rearrangement or other changes including leading overexpression or overactivation of lymphocyte receptor signaling mediator leading to lymphoid malignancies.

In some embodiments, the deregulated lymphocyte receptor signaling is associated with the genetic alterations in B-cell receptor signaling mediator. In some embodiments, the deregulated B-cell receptor signaling mediator includes mutation (loss of function or a deleterious or activating), a translocation, an amplification, or a genomic rearrangement or other changes including leading to over expression or overactivation of B-cell receptor signaling mediator. In certain embodiments, the BCR signaling mediator is CD79, BTK, MALT1, BCL-10, BCL2, TRAF2, TRAF6, TAK1, CARD9, CARD10 (or CARMA3), CARD11 (or CARMA1), CARD14 (or CARMA2), TAB1, TAB2, TAB3, TAK1, IKKα, IKKβ, IKKγ AP11, AP12, AP13, AP14 or A20.

In certain embodiments, the BCR signaling mediator is CD79, BTK, MALT1, BCL-10, BCL2, TRAF2, TRAF6, TAK1, CARD10 (or CARMA3), CARD11 (or CARMA1), CARD14 (or CARMA2), TAB1, TAB2, TAB3, TAK1, IKKα, IKKβ, IKKγ or A20. In further embodiments, the BCR signaling mediator is CD79, BTK, MALT1, BCL-10, BCL2, TRAF2, TRAF6, TAK1, CARD11 (or CARMA1), CARD14 (or CARMA2), TAK1, IKKα, IKKβ, IKKγ or A20.

In further embodiments, the deregulated B-cell receptor (BCR) signaling pathway is further associated with the genetic alterations in IKBKB, NFKBIA, NFKBIE, TNFAIP3, TRAF3, TRAF2, BIRC3, MAP3K14, IKK complex, CBM complex, NF-ϰB target genes or MAPK target genes. In certain embodiments, the deregulated B-cell receptor (BCR) signaling pathway is further associated with the genetic alterations in IKBKB, NFKBIA, NFKBIE, TNFAIP3, TRAF3, TRAF2, BIRC3, MAP3K14, IKK complex, CBM complex or NF-ϰB target genes. In some embodiments, the deregulated B-cell receptor (BCR) signaling pathway is further associated with the alterations in TCF3 genes or ID3 genes.

In certain embodiments, the BCR signaling stimulation results through micro environmental contacts between tumor cells and antigens as suggested by molecular and functional evidences.

In certain embodiments, lymphocyte receptor signaling is T-cell receptor signaling (TCR). In certain embodiments, deregulated lymphocyte receptor signaling is a deregulated T-cell receptor signaling (TCR). In some embodiments, the deregulated lymphocyte receptor signaling is associated with the genetic alterations in T-cell receptor signaling mediator. In some embodiments, the deregulated T-cell receptor signaling mediator includes mutation (loss of function or a deleterious or activating), a translocation, an amplification, or a genomic rearrangement or other changes including leading to overexpression or over activation of T-cell receptor signaling mediator.

In certain embodiments, TCR signaling mediator is FYN, ITK, SYK, PLC-gamma, MALT1, BCL-10, BCL2, TRAF2, TRAF6, TAK1, CARDS, CARD10 (or CARMA3), CARD11 (or CARMA1), CARD14 (or CARMA2), FABP5, TAB1, TAB2, TAB3, TAK1, IKKα, IKKβ, IKKγ, AP11, AP12, AP13, AP14 or A20.

In certain embodiment, the present invention provides a method of inhibiting cancer cell proliferation associated with a deregulated B-cell receptor signaling pathway, comprising contacting the cell with a fatty acid-binding protein 5 (FABP5) inhibitor. In certain preferred embodiment, the present invention provides a method of inhibiting cancer cell proliferation associated with a deregulated B-cell receptor signaling pathway, comprising contacting the cell with a compound of formula (I) or a pharmaceutically acceptable salt or a stereoisomer thereof.

In certain embodiment, the present invention provides a method of inhibiting cancer cell proliferation associated with a deregulated T-cell receptor signaling pathway, comprising contacting the cell with a fatty acid-binding protein 5 (FABP5) inhibitor. In certain preferred embodiment, the present invention provides a method of inhibiting cancer cell proliferation associated with a deregulated T-cell receptor signaling pathway, comprising contacting the cell with a compound of formula (I) or a pharmaceutically acceptable salt or a stereoisomer thereof.

In certain embodiments, the cell is in a subject in need thereof. In certain embodiments, the subject has a cancer characterized by aberrant activity of lymphocyte receptor (e.g., B-cell receptor and T-cell receptor) signaling pathways.

In certain embodiments, the subject has a cancer characterized by aberrant activity of B-cell receptor signaling pathways.

In certain embodiments, the subject has a cancer characterized by aberrant activity of T-cell receptor signaling pathways.

In certain embodiments, contacting the cell occurs in a subject in need thereof, thereby treating a disease or disorder selected from cancer, immune disorders, or immunodeficiency disorders.

In certain embodiments, contacting the cell occurs in a subject in need thereof, thereby treating a cancer associated with a deregulated lymphocyte receptor signaling pathway.

In certain embodiments, the present invention provides a compound represented by formula (I) or a pharmaceutically acceptable salt or a stereoisomer thereof for use as a FABP5 inhibitor in the treatment of a cancer in a subject having associated with a deregulated lymphocyte receptor signaling pathway.

In some embodiments, FABP5 inhibitor of the present invention is the one that covalently and/or irreversibly binds to FABP5. In certain embodiments, FABP5 inhibitor of the present invention binds irreversibly to FABP5 to form a covalent bond. In some embodiments, the subject is treated with covalent and/or irreversible FABP5 inhibitor.

In certain embodiments, FABP5 inhibitors include a-truxillic acid derivatives (as described in Berger et al, PLoS One. 2012; 7(12): e50968), triazolopyrimidinone derivatives (as described in WO2010056631), and cyclobutane derivatives (as described in US201902013).

### Compound of Formula (I)

In certain embodiments, the present invention provides a method of inhibiting cancer cell proliferation associated with a deregulated lymphocyte receptor signaling pathway, comprising contacting the cell with a fatty acid-binding protein 5 (FABP5) inhibitor, wherein the FABP5 inhibitor has the structure of compound of formula (I) or a pharmaceutically acceptable salt or a stereoisomer thereof:

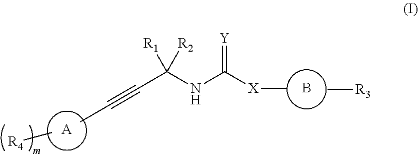

wherein,
A represents aryl or heteroaryl;
X represents N—R, or absent;
Y represents O, S or NCN;
B represents aryl, cycloalkyl or heterocycloalkyl; wherein the aryl, cycloalkyl or heterocycloalkyl are optionally substituted with one or more groups selected from alkyl, halo and oxo;
R₁ represents alkyl; R₂ represents hydrogen or alkyl; or R₁ and R₂ together with the carbon atoms to which they are attached form 3- to 5-membered cycloalkyl ring;
R₃ represents —C(O)Rₐ, —S(O)₂Rₐ, —NHS(O)₂Rₐ, —NR_{b}C(O)Rₐ, ═NOR, heteroaryl, heterocycloalkyl or (heterocycloalkyl)alkyl-; wherein the heteroaryl and heterocycloalkyl are optionally substituted with one or more group selected from alkyl, halo, oxo and —C(O)Rₓ;
R₄ represents alkyl, halo, haloalkyl, cyano, alkoxy, aryloxy, alkoxyaryl, hydroxyalkyl, acetylene, acyl, hydroxy, cycloalkyl or —N(Rₓ)₂; wherein the cycloalkyl is optionally substituted with alkyl;
Rₐ represents alkyl, alkenyl, haloalkyl, cycloalkyl or heterocycloalkyl; wherein the alkyl, alkenyl, haloalkyl, cycloalkyl and heterocycloalkyl are optionally substituted with one or more groups selected from alkyl, halo, aryl, cycloalkyl, haloalkyl, amino, amido, alkylamino, aminoalkyl, hydroxyl, cyano, alkoxy, alkoxyaryl, aryloxy, hydroxyalkyl, carboxylic acid, ester, thioester, oxo(═O) and —C(O)Rₓ;
Rₓ represents hydrogen, alkyl, alkenyl, acyl or —C(O)-cycloalkyl;
R_{y} represents hydrogen or alkyl;
R_{b} represents hydrogen, alkyl or alkenyl;
‘m’ represents 0, 1, 2 or 3.

According to one embodiment, X represents NH. In certain embodiments, X is absent. According to one embodiment, Y represents O. According to one embodiment, A represents aryl. In certain embodiments, A represents phenyl.

In certain embodiments, A represents phenyl which is substituted by ‘m’ occurrences of R₄. In certain embodiments, m represents 1, 2 or 3. In certain particular embodiments, ‘m’ represents 1 or 2.

According to one embodiment, B represents cycloalkyl or heterocycloalkyl optionally substituted with one or more groups selected from alkyl, halo or oxo.

In certain embodiments, B represents cycloalkyl or heterocycloalkyl; wherein heterocycloalkyl is optionally substituted with oxo.

In certain embodiments, B represents heterocycloalkyl. In certain embodiments, B represents 5 to 6-membered heterocycloalkyl. In certain embodiments, B represents

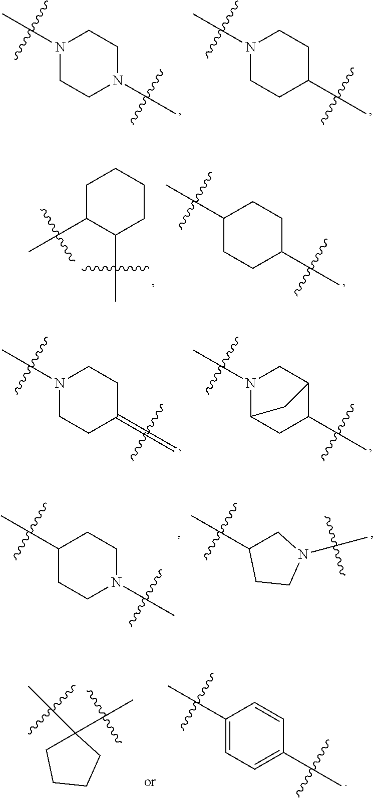

According to one embodiment, R₁ represents alkyl; and R₂ represents hydrogen.

In certain embodiments, R₁ and R₂ together with the carbon atoms to which they are attached form 3 to 5 membered cycloalkyl ring.

In certain embodiments, R₁ and R₂ together with the carbon atoms to which they are attached form cyclopropyl or cyclopentyl ring.

In certain embodiments, R₁ and R₂ together with the carbon atoms to which they are attached form cyclopropyl ring.

According to one embodiment, R₃ represents —C(O)Rₐ, —NHS(O)₂R₁ or —NR_{b}C(O)Rₐ.

According to one embodiment, R₃ represents —C(O)Rₐ; wherein Rₐ is as defined in compound of formula (I).

In certain embodiments, Rₐ represents alkenyl, cycloalkyl or heterocycloalkyl; wherein the alkenyl, cycloalkyl and heterocycloalkyl are optionally substituted with one or more group selected from alkyl, halo, aryl, cycloalkyl, haloalkyl, amino, amido, alkylamino, aminoalkyl, hydroxyl, cyano, alkoxy, alkoxyaryl, aryloxy, hydroxyalkyl, carboxylic acid, ester, thioester or oxo(═O) or —C(O)Rₓ.

According to one embodiment, R₃ represents heterocycloalkyl optionally substituted with —C(O)Rₓ.

In certain embodiments, R_{b} represents hydrogen, or alkyl.

According to one embodiment, R₄ represents alkyl, halo, haloalkyl or cycloalkyl, wherein the cycloalkyl is optionally substituted with alkyl.

In yet another embodiment, FABP5 inhibitor has a structure of compound of formula (IA):

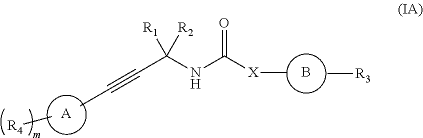

or a pharmaceutically acceptable salt or stereoisomer thereof; wherein A, R₁, R₂, R₃, R₄, B, X and m are as defined in compound of formula (I).

According to one embodiment of compound of formula (IA), X represents NH. According to one embodiment of compound of formula (IA), A represents aryl.

In certain embodiments of compound of formula (IA), A represents phenyl.

According to one embodiment, of compound of formula (IA) or a pharmaceutically acceptable salt or stereoisomer thereof, B represents cycloalkyl or heterocycloalkyl are optionally substituted with one or more groups selected from alkyl, halo or oxo.

According to one embodiment, of compound of formula (IA) or a pharmaceutically acceptable salt or stereoisomer thereof, B represents 5- or 6-membered cycloalkyl. According to one embodiment of compound of formula (IA) or a pharmaceutically acceptable salt or stereoisomer thereof, B represents cyclopentyl or cyclohexyl ring.

According to one embodiment of compound of formula (IA) or a pharmaceutically acceptable salt or stereoisomer thereof, R₃ represents —C(O)Rₐ, —S(O)₂Rₐ, —NHS(O )₂Rₐ, —NR_{b}C(O)Rₐ or ═NORₐ.

According to one embodiment of compound of formula (IA) or a pharmaceutically acceptable salt or stereoisomer thereof, R₃ represents —NHS(O)₂Rₐ or —NR_{b}C(O)Rₐ; wherein Rₐ and R_{b} are as defined in compound of formula (I).

According to one embodiment of compound of formula (IA) or a pharmaceutically acceptable salt or stereoisomer thereof, R₄ represents alkyl, halo, haloalkyl or cycloalkyl, wherein the cycloalkyl is optionally substituted with alkyl.

In yet another embodiment, FABP5 inhibitor has a structure of compound of formula (IB):

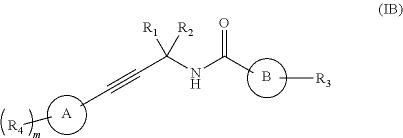

or a pharmaceutically acceptable salt or stereoisomer thereof; wherein A, R₁, R₂, R₃, R₄, B, and m are as defined in compound of formula (I).

According to one embodiment of compound of formula (IB) or a pharmaceutically acceptable salt or stereoisomer thereof, A represents aryl.

According to one embodiment of compound of formula (IB) or a pharmaceutically acceptable salt or stereoisomer thereof, B represents cycloalkyl or heterocycloalkyl are optionally substituted with one or more groups selected from alkyl, halo or oxo.

According to one embodiment of compound of formula (IB) or a pharmaceutically acceptable salt or stereoisomer thereof, B represents heterocycloalkyl optionally substituted with one or more groups selected from alkyl, halo or oxo.

According to one embodiment, of compound of formula (IB) or a pharmaceutically acceptable salt or stereoisomer thereof, B represents 5- or 6-membered heterocycloalkyl.

According to one embodiment of compound of formula (IB) or a pharmaceutically acceptable salt or stereoisomer thereof, R₃ represents heterocycloalkyl optionally substituted with —C(O)Rₓ.

According to one embodiment of compound of formula (IB) or a pharmaceutically acceptable salt or stereoisomer thereof, R₄ represents alkyl, halo, haloalkyl or cycloalkyl, wherein the cycloalkyl is optionally substituted with alkyl.

In yet another embodiment, FABP5 inhibitor has a structure of compound of formula (IC):

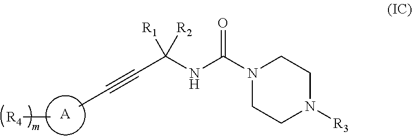

or a pharmaceutically acceptable salt or stereoisomer thereof; wherein A, R₁, R₂, R₃, R₄, and m are as defined in compound of formula (I).

According to one embodiment of compound of formula (IC) or a pharmaceutically acceptable salt or stereoisomer thereof, A represents aryl.

According to one embodiment of compound of formula (IC) or a pharmaceutically acceptable salt or stereoisomer thereof, R₁ represents alkyl; and R₂ represents hydrogen or alkyl.

According to one embodiment of compound of formula (IC) or a pharmaceutically acceptable salt or stereoisomer thereof, R₁ and R₂ together with the carbon atoms to which they are attached form cyclopropyl or cyclopentyl ring.

According to one embodiment of compound of formula (IC) or a pharmaceutically acceptable salt or stereoisomer thereof, R₃ represents optionally substituted heteroaryl, heterocycloalkyl or (heterocycloalkyl)alkyl-.

According to one embodiment of compound of formula (IC) or a pharmaceutically acceptable salt or stereoisomer thereof, R₃ represents heterocycloalkyl optionally substituted with —C(O)Rₓ.

According to one embodiment of compound of formula (IC) or a pharmaceutically acceptable salt or stereoisomer thereof, R₃ represents heterocycloalkyl optionally substituted with —C(O)Rₓ.

According to one embodiment of compound of formula (IC) or a pharmaceutically acceptable salt or stereoisomer thereof, R₄ represents alkyl, halo, haloalkyl or cycloalkyl, wherein the cycloalkyl is optionally substituted with alkyl.

According to one embodiment of compound of formula (IC) or a pharmaceutically acceptable salt or stereoisomer thereof, ‘m’ represents 2.

In yet another embodiment, FABP5 inhibitor has a structure of compound of formula (ID):

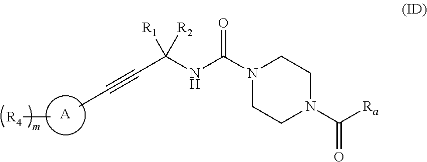

or a pharmaceutically acceptable salt or stereoisomer thereof; wherein A, R₁, R₂, R₄, Rₐ and ‘m’ are as defined in compound of formula (I).

According to one embodiment of compound of formula (ID) or a pharmaceutically acceptable salt or stereoisomer thereof, A represents aryl.

According to one embodiment of compound of formula (ID) or a pharmaceutically acceptable salt or stereoisomer thereof, R₁ represents alkyl; and R₂ independently represents hydrogen.

According to one embodiment of compound of formula (ID) or a pharmaceutically acceptable salt or stereoisomer thereof, Rₐ represents alkenyl, cycloalkyl or heterocycloalkyl; wherein the alkenyl, cycloalkyl and heterocycloalkyl are optionally substituted with one or more groups selected from halo, aryl, haloalkyl or carboxylic acid.

According to one embodiment of compound of formula (ID) or a pharmaceutically acceptable salt or stereoisomer thereof, Rₐ represents represents alkenyl substituted with alkyl or haloalkyl.

According to one embodiment of compound of formula (ID) or a pharmaceutically acceptable salt or stereoisomer thereof, R₄ represents alkyl, halo, haloalkyl or cycloalkyl, wherein the cycloalkyl is optionally substituted with alkyl.

According to one embodiment of compound of formula (ID) or a pharmaceutically acceptable salt or stereoisomer thereof, R₄ represents halo.

According to one embodiment of compound of formula (ID) or a pharmaceutically acceptable salt or stereoisomer thereof, m represents 2.

In yet another embodiment, FABP5 inhibitor has a structure of compounds of formula (IE):

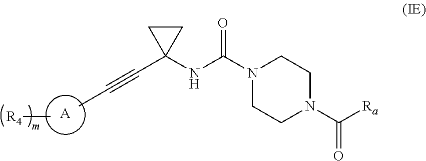

or a pharmaceutically acceptable salt or stereoisomer thereof; wherein A, R₄, Rₐ and m are as defined in compound of formula (I).

According to one embodiment of compound of formula (IE) or a pharmaceutically acceptable salt or stereoisomer thereof, A represents aryl.

According to one embodiment of compound of formula (IE) or a pharmaceutically acceptable salt or stereoisomer thereof, Ra represents alkenyl, cycloalkyl or heterocycloalkyl; wherein the alkenyl, cycloalkyl and heterocycloalkyl are optionally substituted with one or more groups selected from halo, aryl, haloalkyl or carboxylic acid.

According to one embodiment of compound of formula (IE) or a pharmaceutically acceptable salt or stereoisomer thereof, R₄ represents halo.

According to one embodiment of compound of formula (IE) or a pharmaceutically acceptable salt or stereoisomer thereof, m represents 2.

In yet another embodiment, FABP5 inhibitor has a structure of compound of formula (IF):

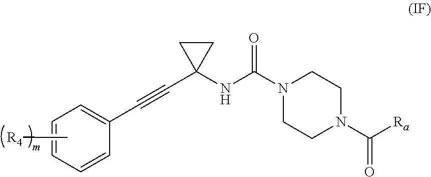

or a pharmaceutically acceptable salt or stereoisomer thereof; wherein R₄, Rₐ and m are as defined in compound of formula (I).

According to one embodiment of compound of formula (IF) or a pharmaceutically acceptable salt or stereoisomer thereof, Rₐ represents alkenyl, cycloalkyl or heterocycloalkyl; wherein the alkenyl, cycloalkyl and heterocycloalkyl are optionally substituted with one or more groups selected from halo, aryl, haloalkyl or carboxylic acid.

According to one embodiment of compound of formula (IF) or a pharmaceutically acceptable salt or stereoisomer thereof, R₄ represents halo.

According to one embodiment of compound of formula (IF) or a pharmaceutically acceptable salt or stereoisomer thereof, m represents 2.

In yet another embodiment, FABP5 inhibitor has a structure of compound of formula (IG):

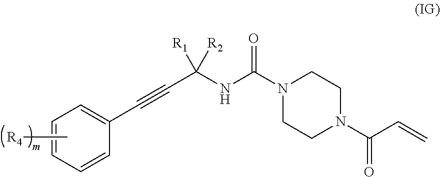

or a pharmaceutically acceptable salt or stereoisomer thereof; wherein R₁, R₂, R₄ and m are as defined in compound of formula (I).

According to one embodiment of compound of formula (IG) or a pharmaceutically acceptable salt or stereoisomer thereof, R₁ represents alkyl; and R₂ independently represents hydrogen.

According to one embodiment of compound of formula (IG) or a pharmaceutically acceptable salt or stereoisomer thereof, R₄ represents halo.

According to one embodiment of compound of formula (IG) or a pharmaceutically acceptable salt or stereoisomer thereof, m represents 2.

In yet another embodiment, FABP5 inhibitor has a structure of compound of formula (IH):

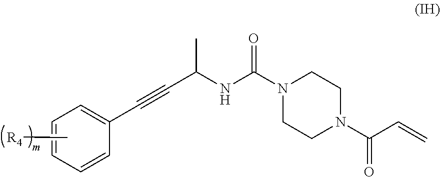

or a pharmaceutically acceptable salt or stereoisomer thereof; wherein R₄ and m are as defined in compound of formula (I).

According to one embodiment of compound of formula (IH) or a pharmaceutically acceptable salt or stereoisomer thereof, R₄ represents halo.

According to one embodiment of compound of formula (IH) or a pharmaceutically acceptable salt or stereoisomer thereof, R₄ represents chloro.

According to one embodiment of compound of formula (IH) or a pharmaceutically acceptable salt or stereoisomer thereof, m represents 2.

In certain embodiment, FABP5 inhibitor of the present invention has a structure of compound of formula (IA), compound of formula (IB), compound of formula (IC), compound of formula (ID), compound of formula (IE), compound of formula (IF), compound of formula (IG), or compound of formula (IG); or a pharmaceutically acceptable salt or a stereoisomer thereof.

In certain embodiments, the present invention provides a method of inhibiting cancer cell proliferation associated with a deregulated lymphocyte receptor signaling pathway, comprising contacting the cell with any of compound of formula (IA), compound of formula (IB), compound of formula (IC), compound of formula (ID), compound of formula (IE), compound of formula (IF), compound of formula (IG), or compound of formula (IG); or a pharmaceutically acceptable salt or a stereoisomer thereof.

According to yet another embodiment, FABP5 inhibitor comprises a compound, or a pharmaceutically acceptable salt or a stereoisomer thereof, selected from:

TABLE - I

| Compound | Structure |
|---|---|
| 1 | 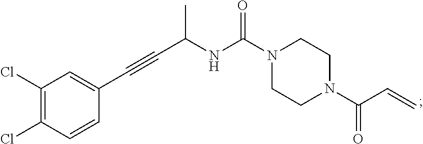 [SMILES: C=CC(=O)N1CCN(C(=O)NC(C)C#Cc2ccc(Cl)c(Cl)c2)CC1] |
| 1a | 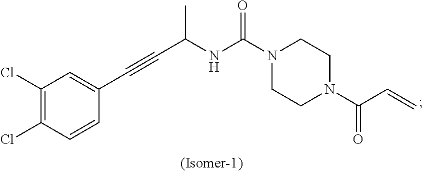 [SMILES: C=CC(=O)N1CCN(C(=O)NC(C)C#Cc2ccc(Cl)c(Cl)c2)CC1] |
| 1b | 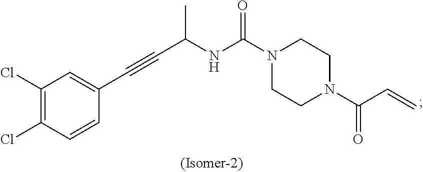 [SMILES: C=CC(=O)N1CCN(C(=O)NC(C)C#Cc2ccc(Cl)c(Cl)c2)CC1] |
| 2 | 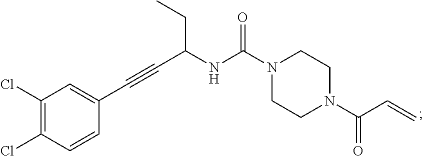 [SMILES: C=CC(=O)N1CCN(C(=O)NC(C#Cc2ccc(Cl)c(Cl)c2)CC)CC1] |
| 2a | 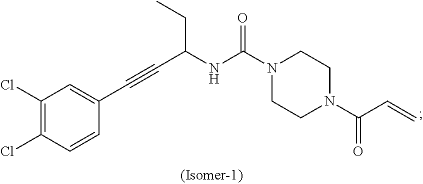 [SMILES: C=CC(=O)N1CCN(C(=O)NC(C#Cc2ccc(Cl)c(Cl)c2)CC)CC1] |
| 2b | 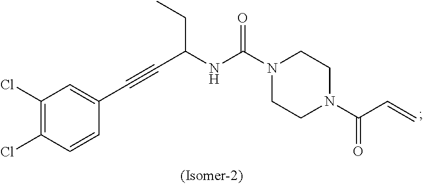 [SMILES: C=CC(=O)N1CCN(C(=O)NC(C#Cc2ccc(Cl)c(Cl)c2)CC)CC1] |
| 3 | 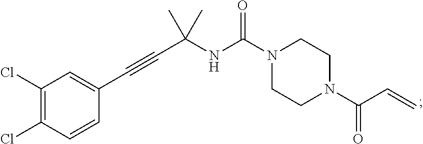 [SMILES: C=CC(=O)N1CCN(C(=O)NC(C)(C)C#Cc2ccc(Cl)c(Cl)c2)CC1] |
| 4 | 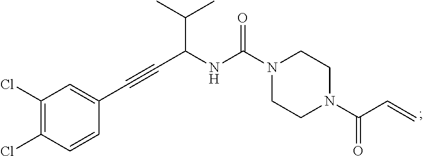 [SMILES: C=CC(=O)N1CCN(C(=O)NC(C#Cc2ccc(Cl)c(Cl)c2)C(C)C)CC1] |
| 5 | 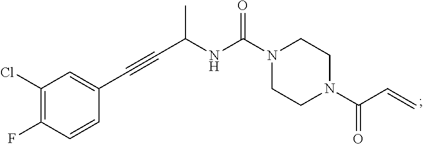 [SMILES: C=CC(=O)N1CCN(C(=O)NC(C)C#Cc2ccc(F)c(Cl)c2)CC1] |
| 6 | 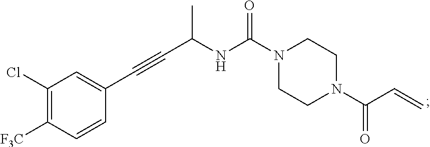 [SMILES: C=CC(=O)N1CCN(C(=O)NC(C)C#Cc2ccc(C(F)(F)F)c(Cl)c2)CC1] |
| 7 | 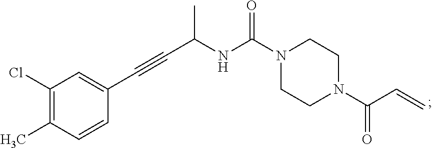 [SMILES: C=CC(=O)N1CCN(C(=O)NC(C)C#Cc2ccc(C)c(Cl)c2)CC1] |
| 8 | 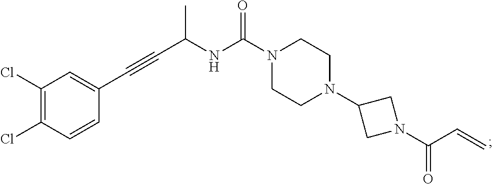 [SMILES: C=CC(=O)N1CC(N2CCN(C(=O)NC(C)C#Cc3ccc(Cl)c(Cl)c3)CC2)C1] |
| 9 | 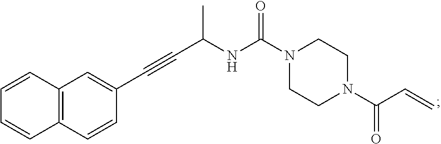 [SMILES: C=CC(=O)N1CCN(C(=O)NC(C)C#Cc2ccc3ccccc3c2)CC1] |
| 10 | 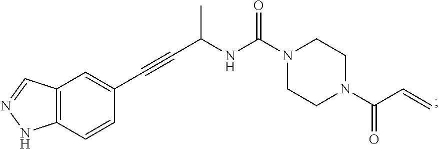 [SMILES: C=CC(=O)N1CCN(C(=O)NC(C)C#Cc2ccc3[nH]ncc3c2)CC1] |
| 11 | 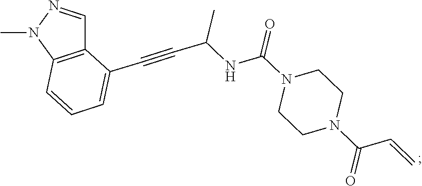 [SMILES: C=CC(=O)N1CCN(C(=O)NC(C)C#Cc2cccc3c2cnn3C)CC1] |
| 12 | 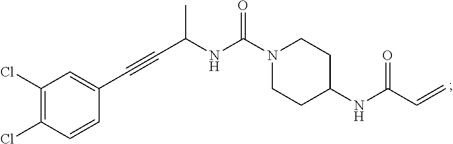 [SMILES: C=CC(=O)NC1CCN(C(=O)NC(C)C#Cc2ccc(Cl)c(Cl)c2)CC1] |
| 13 | 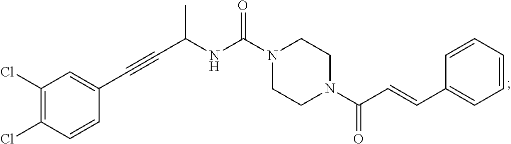 [SMILES: CC(C#Cc1ccc(Cl)c(Cl)c1)NC(=O)N1CCN(C(=O)/C=C/c2ccccc2)CC1] |
| 14 | 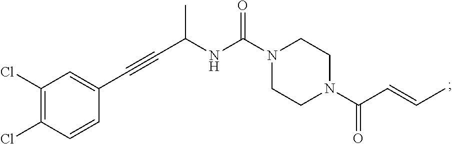 [SMILES: C/C=C/C(=O)N1CCN(C(=O)NC(C)C#Cc2ccc(Cl)c(Cl)c2)CC1] |
| 15 | 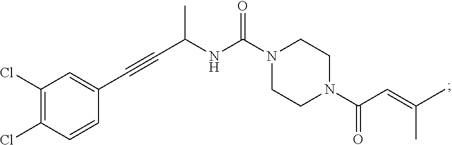 [SMILES: CC(C)=CC(=O)N1CCN(C(=O)NC(C)C#Cc2ccc(Cl)c(Cl)c2)CC1] |
| 16 | 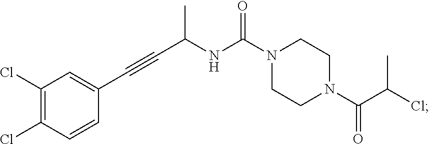 [SMILES: CC(C#Cc1ccc(Cl)c(Cl)c1)NC(=O)N1CCN(C(=O)C(C)Cl)CC1] |
| 17 | 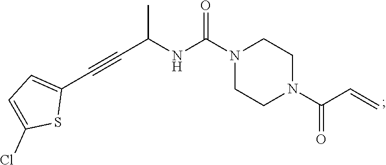 [SMILES: C=CC(=O)N1CCN(C(=O)NC(C)C#Cc2ccc(Cl)s2)CC1] |
| 18 | 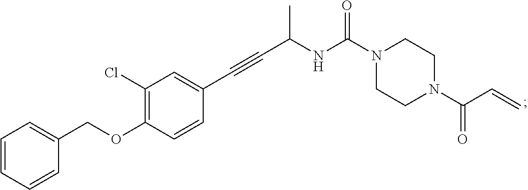 [SMILES: C=CC(=O)N1CCN(C(=O)NC(C)C#Cc2ccc(OCc3ccccc3)c(Cl)c2)CC1] |
| 19 | 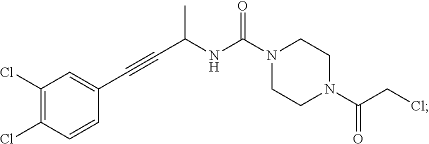 [SMILES: CC(C#Cc1ccc(Cl)c(Cl)c1)NC(=O)N1CCN(C(=O)CCl)CC1] |
| 20 | 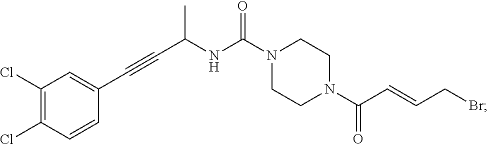 [SMILES: CC(C#Cc1ccc(Cl)c(Cl)c1)NC(=O)N1CCN(C(=O)/C=C/CBr)CC1] |
| 21 | 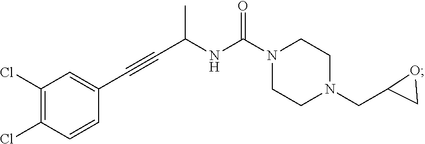 [SMILES: CC(C#Cc1ccc(Cl)c(Cl)c1)NC(=O)N1CCN(CC2CO2)CC1] |
| 22 | 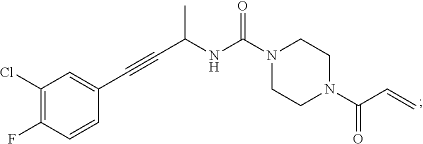 [SMILES: C=CC(=O)N1CCN(C(=O)NC(C)C#Cc2ccc(F)c(Cl)c2)CC1] |
| 23 | 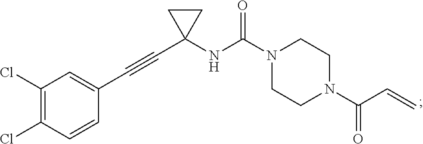 [SMILES: C=CC(=O)N1CCN(C(=O)NC2(C#Cc3ccc(Cl)c(Cl)c3)CC2)CC1] |
| 24 | 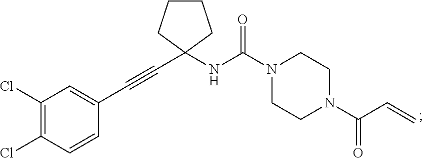 [SMILES: C=CC(=O)N1CCN(C(=O)NC2(C#Cc3ccc(Cl)c(Cl)c3)CCCC2)CC1] |
| 25 | 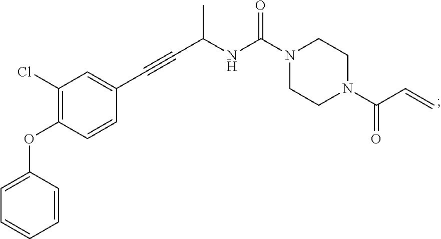 [SMILES: C=CC(=O)N1CCN(C(=O)NC(C)C#Cc2ccc(Oc3ccccc3)c(Cl)c2)CC1] |
| 26 | 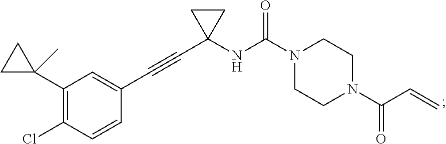 [SMILES: C=CC(=O)N1CCN(C(=O)NC2(C#Cc3ccc(Cl)c(C4(C)CC4)c3)CC2)CC1] |
| 27 | 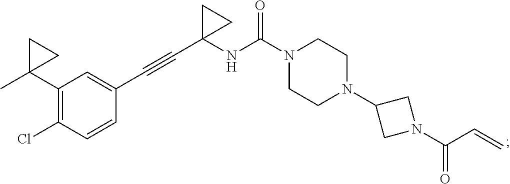 [SMILES: C=CC(=O)N1CC(N2CCN(C(=O)NC3(C#Cc4ccc(Cl)c(C5(C)CC5)c4)CC3)CC2)C1] |
| 28 | 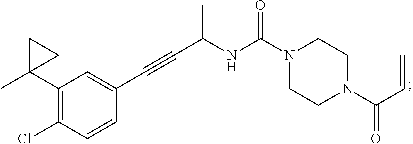 [SMILES: C=CC(=O)N1CCN(C(=O)NC(C)C#Cc2ccc(Cl)c(C3(C)CC3)c2)CC1] |
| 29 | 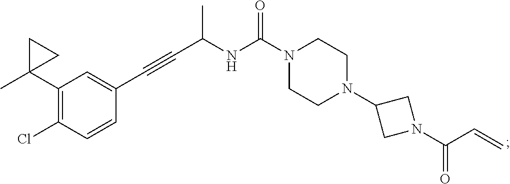 [SMILES: C=CC(=O)N1CC(N2CCN(C(=O)NC(C)C#Cc3ccc(Cl)c(C4(C)CC4)c3)CC2)C1] |
| 30 | 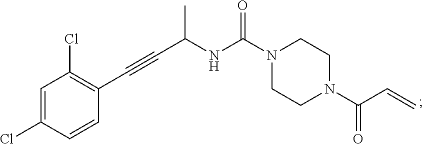 [SMILES: C=CC(=O)N1CCN(C(=O)NC(C)C#Cc2ccc(Cl)cc2Cl)CC1] |
| 31 | 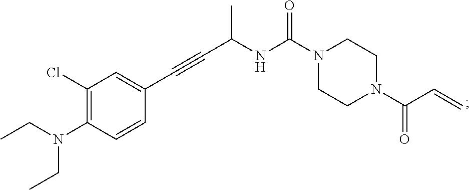 [SMILES: C=CC(=O)N1CCN(C(=O)NC(C)C#Cc2ccc(N(CC)CC)c(Cl)c2)CC1] |
| 32 | 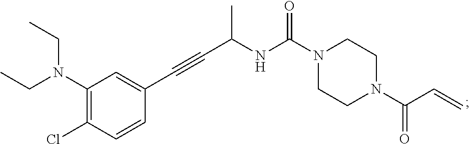 [SMILES: C=CC(=O)N1CCN(C(=O)NC(C)C#Cc2ccc(Cl)c(N(CC)CC)c2)CC1] |
| 33 | 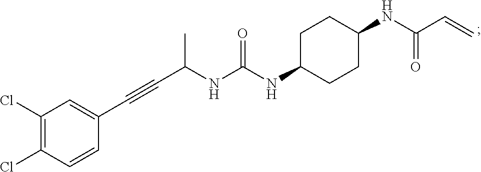 [SMILES: C=CC(=O)N[C@H]1CC[C@@H](NC(=O)NC(C)C#Cc2ccc(Cl)c(Cl)c2)CC1] |
| 34 | 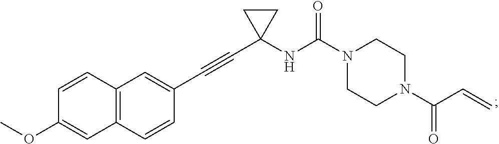 [SMILES: C=CC(=O)N1CCN(C(=O)NC2(C#Cc3ccc4cc(OC)ccc4c3)CC2)CC1] |
| 35 | 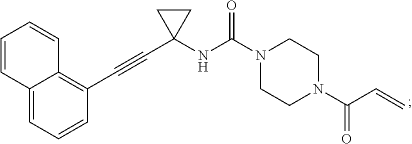 [SMILES: C=CC(=O)N1CCN(C(=O)NC2(C#Cc3cccc4ccccc34)CC2)CC1] |
| 36 | 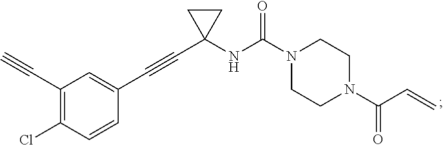 [SMILES: C#Cc1cc(C#CC2(NC(=O)N3CCN(C(=O)C=C)CC3)CC2)ccc1Cl] |
| 37 | 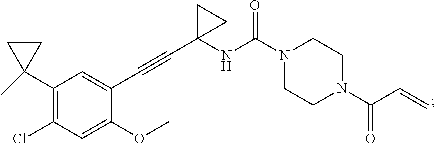 [SMILES: C=CC(=O)N1CCN(C(=O)NC2(C#Cc3cc(C4(C)CC4)c(Cl)cc3OC)CC2)CC1] |
| 38 | 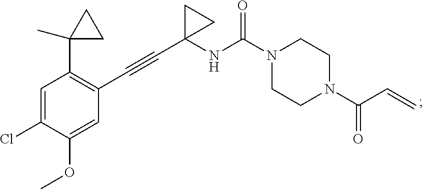 [SMILES: C=CC(=O)N1CCN(C(=O)NC2(C#Cc3cc(OC)c(Cl)cc3C3(C)CC3)CC2)CC1] |
| 39 | 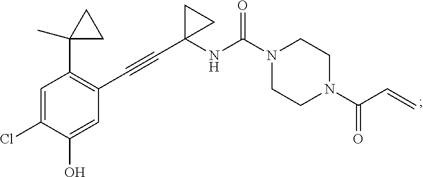 [SMILES: C=CC(=O)N1CCN(C(=O)NC2(C#Cc3cc(O)c(Cl)cc3C3(C)CC3)CC2)CC1] |
| 40 | 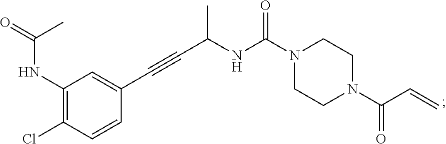 [SMILES: C=CC(=O)N1CCN(C(=O)NC(C)C#Cc2ccc(Cl)c(NC(C)=O)c2)CC1] |
| 41 | 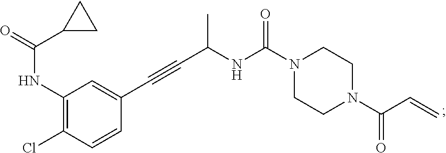 [SMILES: C=CC(=O)N1CCN(C(=O)NC(C)C#Cc2ccc(Cl)c(NC(=O)C3CC3)c2)CC1] |
| 42 | 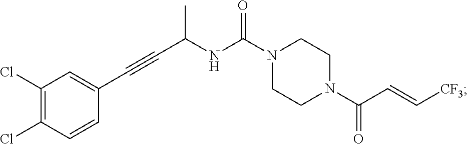 [SMILES: CC(C#Cc1ccc(Cl)c(Cl)c1)NC(=O)N1CCN(C(=O)/C=C/C(F)(F)F)CC1] |
| 42a | 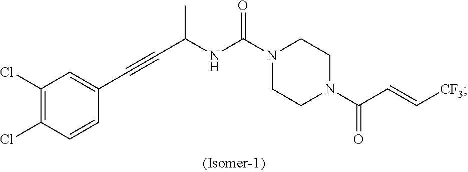 [SMILES: CC(C#Cc1ccc(Cl)c(Cl)c1)NC(=O)N1CCN(C(=O)/C=C/C(F)(F)F)CC1] |
| 42b | 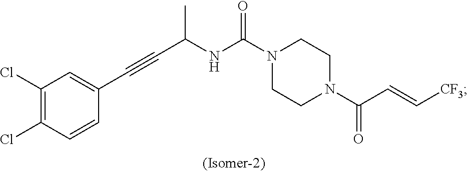 [SMILES: CC(C#Cc1ccc(Cl)c(Cl)c1)NC(=O)N1CCN(C(=O)/C=C/C(F)(F)F)CC1] |
| 43 | 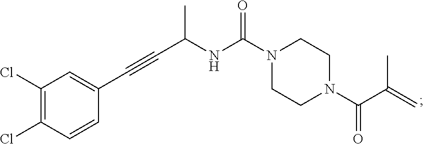 [SMILES: C=C(C)C(=O)N1CCN(C(=O)NC(C)C#Cc2ccc(Cl)c(Cl)c2)CC1] |
| 44 | 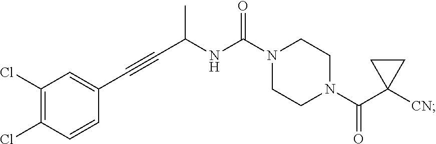 [SMILES: CC(C#Cc1ccc(Cl)c(Cl)c1)NC(=O)N1CCN(C(=O)C2(C#N)CC2)CC1] |
| 45 | 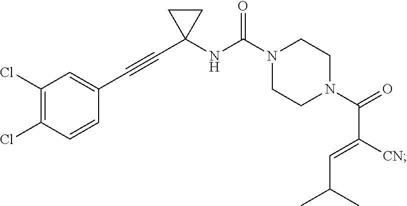 [SMILES: CC(C)/C=C(\C#N)C(=O)N1CCN(C(=O)NC2(C#Cc3ccc(Cl)c(Cl)c3)CC2)CC1] |
| 46 | 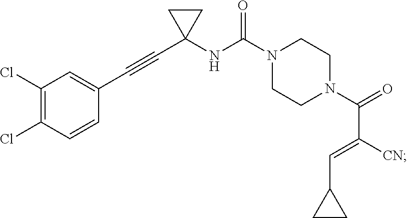 [SMILES: N#C/C(=C\C1CC1)C(=O)N1CCN(C(=O)NC2(C#Cc3ccc(Cl)c(Cl)c3)CC2)CC1] |
| 47 | 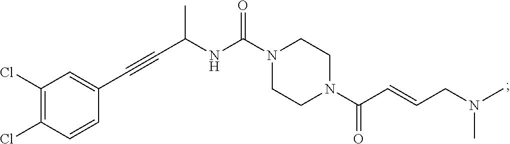 [SMILES: CC(C#Cc1ccc(Cl)c(Cl)c1)NC(=O)N1CCN(C(=O)/C=C/CN(C)C)CC1] |
| 48a | 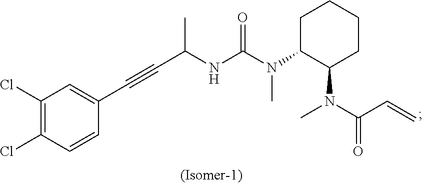 [SMILES: C=CC(=O)N(C)[C@@H]1CCCC[C@H]1N(C)C(=O)NC(C)C#Cc1ccc(Cl)c(Cl)c1] |
| 48b | 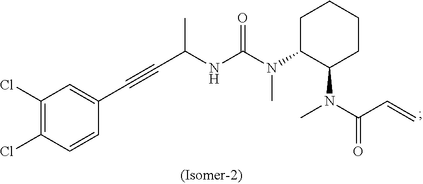 [SMILES: C=CC(=O)N(C)[C@@H]1CCCC[C@H]1N(C)C(=O)NC(C)C#Cc1ccc(Cl)c(Cl)c1] |
| 49 | 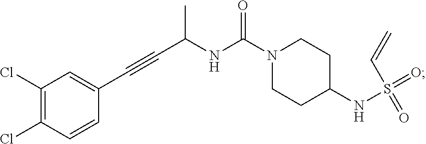 [SMILES: C=CS(=O)(=O)NC1CCN(C(=O)NC(C)C#Cc2ccc(Cl)c(Cl)c2)CC1] |
| 50 | 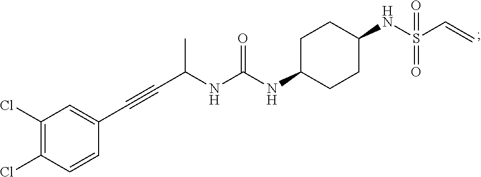 [SMILES: C=CS(=O)(=O)N[C@H]1CC[C@@H](NC(=O)NC(C)C#Cc2ccc(Cl)c(Cl)c2)CC1] |
| 51 | 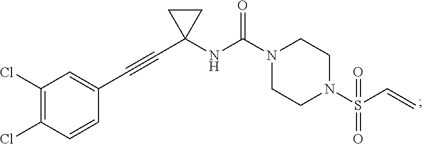 [SMILES: C=CS(=O)(=O)N1CCN(C(=O)NC2(C#Cc3ccc(Cl)c(Cl)c3)CC2)CC1] |
| 52 | 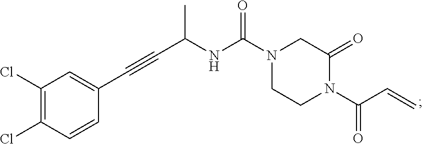 [SMILES: C=CC(=O)N1CCN(C(=O)NC(C)C#Cc2ccc(Cl)c(Cl)c2)CC1=O] |
| 53 | 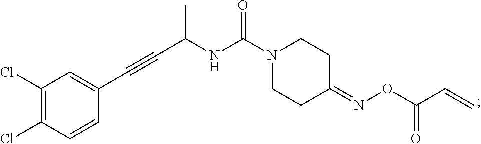 [SMILES: C=CC(=O)ON=C1CCN(C(=O)NC(C)C#Cc2ccc(Cl)c(Cl)c2)CC1] |
| 54 | 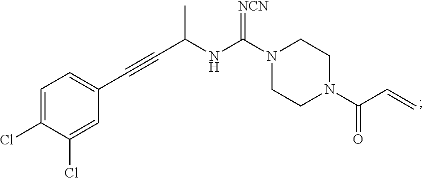 |
| 55 | 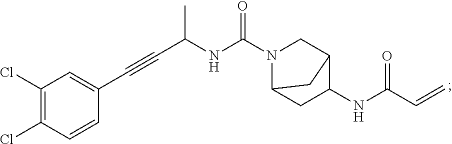 [SMILES: C=CC(=O)NC1CC2CC1CN2C(=O)NC(C)C#Cc1ccc(Cl)c(Cl)c1] |
| 56 | 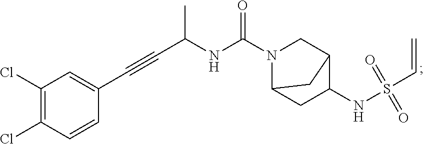 [SMILES: C=CS(=O)(=O)NC1CC2CC1CN2C(=O)NC(C)C#Cc1ccc(Cl)c(Cl)c1] |
| 57 | 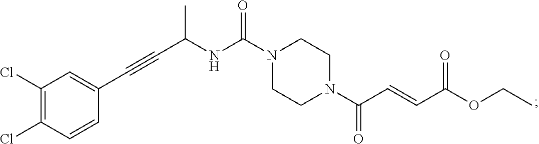 [SMILES: CCOC(=O)/C=C/C(=O)N1CCN(C(=O)NC(C)C#Cc2ccc(Cl)c(Cl)c2)CC1] |
| 58 | 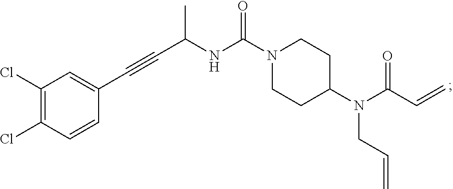 [SMILES: C=CCN(C(=O)C=C)C1CCN(C(=O)NC(C)C#Cc2ccc(Cl)c(Cl)c2)CC1] |
| 59 | 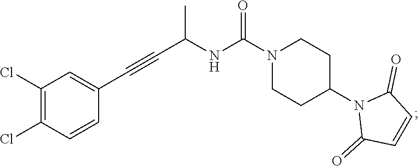 [SMILES: CC(C#Cc1ccc(Cl)c(Cl)c1)NC(=O)N1CCC(N2C(=O)C=CC2=O)CC1] |
| 60 | 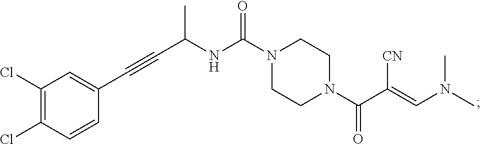 [SMILES: CC(C#Cc1ccc(Cl)c(Cl)c1)NC(=O)N1CCN(C(=O)/C(C#N)=C/N(C)C)CC1] |
| 61 | 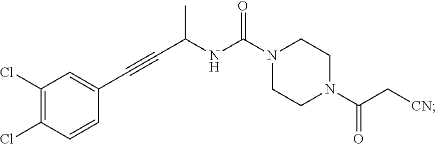 [SMILES: CC(C#Cc1ccc(Cl)c(Cl)c1)NC(=O)N1CCN(C(=O)CC#N)CC1] |
| 62 | 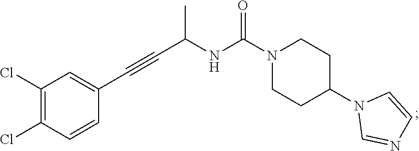 [SMILES: CC(C#Cc1ccc(Cl)c(Cl)c1)NC(=O)N1CCC(n2ccnc2)CC1] |
| 63 | 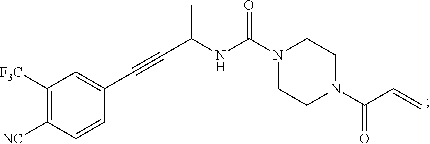 [SMILES: C=CC(=O)N1CCN(C(=O)NC(C)C#Cc2ccc(C#N)c(C(F)(F)F)c2)CC1] |
| 64 | 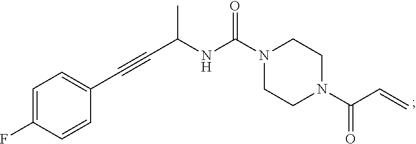 [SMILES: C=CC(=O)N1CCN(C(=O)NC(C)C#Cc2ccc(F)cc2)CC1] |
| 65 | 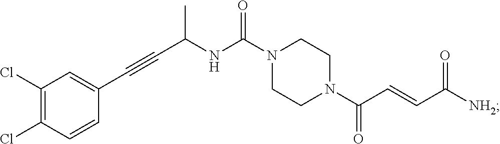 [SMILES: CC(C#Cc1ccc(Cl)c(Cl)c1)NC(=O)N1CCN(C(=O)/C=C/C(N)=O)CC1] |
| 66 | 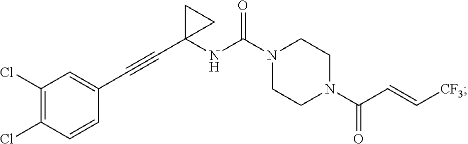 [SMILES: O=C(/C=C/C(F)(F)F)N1CCN(C(=O)NC2(C#Cc3ccc(Cl)c(Cl)c3)CC2)CC1] |
| 67 | 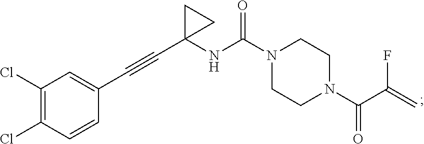 [SMILES: C=C(F)C(=O)N1CCN(C(=O)NC2(C#Cc3ccc(Cl)c(Cl)c3)CC2)CC1] |
| 68 | 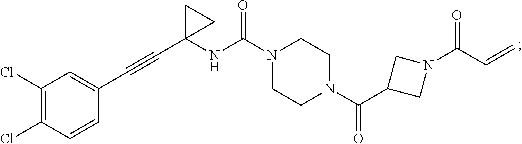 [SMILES: C=CC(=O)N1CC(C(=O)N2CCN(C(=O)NC3(C#Cc4ccc(Cl)c(Cl)c4)CC3)CC2)C1] |
| 69 | 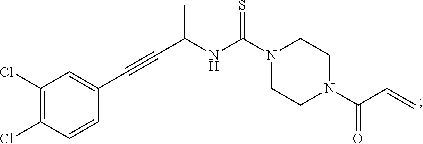 [SMILES: C=CC(=O)N1CCN(C(=S)NC(C)C#Cc2ccc(Cl)c(Cl)c2)CC1] |
| 70 | 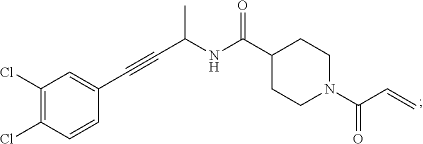 [SMILES: C=CC(=O)N1CCC(C(=O)NC(C)C#Cc2ccc(Cl)c(Cl)c2)CC1] |
| 71 | 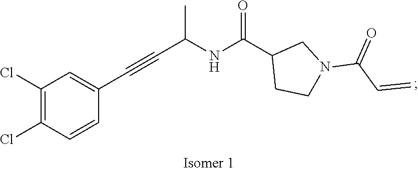 [SMILES: C=CC(=O)N1CCC(C(=O)NC(C)C#Cc2ccc(Cl)c(Cl)c2)C1] |
| 72 | 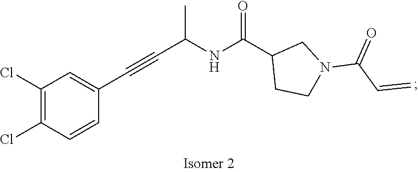 [SMILES: C=CC(=O)N1CCC(C(=O)NC(C)C#Cc2ccc(Cl)c(Cl)c2)C1] |
| 73 | 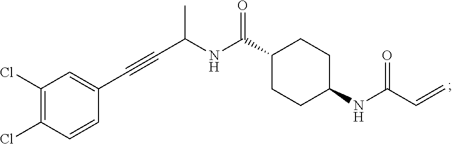 [SMILES: C=CC(=O)N[C@H]1CC[C@H](C(=O)NC(C)C#Cc2ccc(Cl)c(Cl)c2)CC1] |
| 74 | 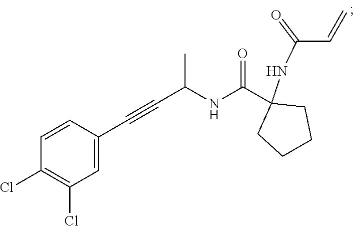 [SMILES: C=CC(=O)NC1(C(=O)NC(C)C#Cc2ccc(Cl)c(Cl)c2)CCCC1] |
| 75 | 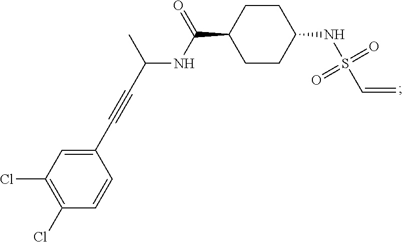 [SMILES: C=CS(=O)(=O)N[C@H]1CC[C@H](C(=O)NC(C)C#Cc2ccc(Cl)c(Cl)c2)CC1] |
| 76 | 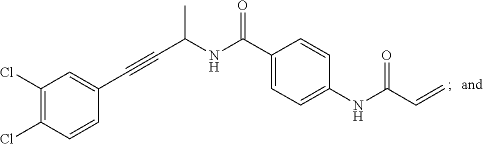 [SMILES: C=CC(=O)Nc1ccc(C(=O)NC(C)C#Cc2ccc(Cl)c(Cl)c2)cc1] |
| 77 | 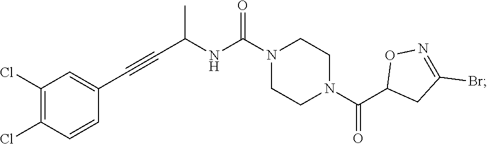 [SMILES: CC(C#Cc1ccc(Cl)c(Cl)c1)NC(=O)N1CCN(C(=O)C2CC(Br)=NO2)CC1] |

In certain embodiments, the present invention provides method of inhibiting cancer cell proliferation associated with a deregulated lymphocyte receptor signaling pathway, comprising contacting the cell with any of the compound of mentioned in Table-I or a pharmaceutically acceptable salt or a stereoisomer thereof.

### Method of Treatment

In some embodiments, the disclosure provides uses of fatty acid-binding protein 5 (FABP5) inhibitor as described herein in modulating deregulated lymphocyte receptor signaling pathway.

In certain embodiments, the disclosure provides uses of fatty acid-binding protein 5 (FABP5) inhibitor as described herein in inhibiting cancer cell proliferation associated with a deregulated lymphocyte receptor signaling pathway.

In certain embodiments, the present invention provides a method of treating cancer in a subject having a deregulated lymphocyte receptor signaling pathway, comprising administering to the subject in need thereof a a therapeutically effective amount of a fatty acid-binding protein 5 (FABP5) inhibitor as described herein or a pharmaceutically acceptable acceptable salt thereof. In certain embodiments, the present invention provides a method of treating cancer in a subject having a deregulated lymphocyte receptor signaling pathway, comprising administering the subject in need thereof a compound of Formula (I), or a pharmaceutically acceptable salt or a stereoisomer thereof, according to any of the above embodiments.

In certain embodiments, the disease or disorder is cancer. In some embodiments, the cancer is a hematologic cancer. In certain embodiments, the cancer is B-cell cancer or T-cell cancer. In certain embodiments, the treatment of a disease or disorder comprises inhibiting growth of B-cell tumor cells, T-cell tumor cells and/or metastasis.

In some embodiments, cancer is selected from among a leukemia, a lymphoma, or a myeloma. In certain embodiments, the cancer is B-cell cancer. In certain embodiments, the present invention provides a method of treating B-cell cancer in a subject having a deregulated B-cell receptor signaling pathway, comprising administering the subject in need thereof a compound of Formula (I), or a pharmaceutically acceptable salt or a stereoisomer thereof, according to any of the above embodiments.

In some embodiments, the B-cell cancer is chronic lymphocytic leukemia (CLL), small lymphocytic lymphoma (SLL), diffuse large B-cell lymphoma (DLBCL), activated B-cell diffuse large B-cell lymphoma (ABC-DLBCL), germinal center diffuse large B-cell lymphoma (GCB DLBCL), primary mediastinal B-cell lymphoma (PMBL), non-Hodgkin lymphoma, Burkitt's lymphoma, follicular lymphoma, immunoblastic large cell lymphoma, precursor B-lymphoblastic lymphoma, precursor B-cell acute lymphoblastic leukemia, hairy cell leukemia, mantle cell lymphoma, B cell prolymphocytic leukaemia, lymphoplasmacytic lymphoma/Waldenstrom macroglobulinemia, splenic marginal zone lymphoma, plasma cell myeloma, plasmacytoma, extranodal marginal zone B cell lymphoma, nodal marginal zone B cell lymphoma, mediastinal (thymic) large B cell lymphoma, intravascular large B cell lymphoma, primary effusion lymphoma, or lymphomatoid granulomatosis.

In certain embodiments, B-cell cancer is a non-Hodgkin's lymphoma, a Hodgkin's lymphoma, a chronic lymphocytic leukaemia (CLL) or a multiple myeloma. In certain embodiments, non-Hodgkin's lymphoma is a follicular lymphoma, a diffuse large B cell lymphoma (DLBCL) of activated B cell (ABC) type, a diffuse large B cell lymphoma (DLBCL) of germinal center B cell (GCB) type, a mantle zone lymphoma (MZL), Mantle cell lymphoma (MCL), Primary mediastinal B-cell lymphoma (PMBCL), Waldenstrom macroglobulinemia, Burkitt lymphoma or MALT Lymphoma.

In certain embodiments, B-cell cancer is CLL.

In certain embodiments, the cancer is T-cell cancer. In certain embodiments, the present invention provides a method of treating T-cell cancer in a subject having a deregulated T-cell receptor signaling pathway, comprising administering the subject in need thereof a compound of Formula (I), or a pharmaceutically acceptable salt or a stereoisomer thereof, according to any of the above embodiments.

In some embodiments, T-cell cancer is T cell leukemia or T-cell lymphoma. In certain embodiments, T-cell malignancy is peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or a treatment-related T-cell lymphoma. In certain embodiments, T cell cancer is T-cell acute lymphoblastic leukaemia (T-ALL), peripheral T-cell lymphoma (PTCL), T-cell lymphoblastic lymphoma (T-CLL), cutaneous T-cell lymphoma (CTCL) or adult T-cell lymphoma (ATCL).

In certain embodiments, the invention includes inhibiting the growth of a solid tumor by contacting the tumor with a FABP5 inhibitor. In certain embodiments, the present invention provides a method of treating solid tumor in a subject comprising administering a subject in need thereof a therapeutically effective amount of a FABP5 inhibitor. The solid tumour can be a tumour of the prostate, brain, head and neck, cervix, colon, pancreas, bladder, gastric, skin, esophagus, liver, bile duct or kidney.

In certain embodiments, the present invention provides a use of FABP5 inhibitors in the manufacture of medicament for inhibiting cancer cell proliferation associated with a deregulated lymphocyte receptor signaling pathway.

### Pharmaceutical Composition

The pharmaceutical composition may be administered by oral or inhalation routes, or by parenteral administration route. For example, compositions can be administered orally, by intravenous infusion, topically, intraperitoneally, intravesically, intrathecally, or as a suppository. Examples of parenteral administration includes but not limited to intraarticular (in the joints), intravenous, intramuscular, intradermal, intraperitoneal, and subcutaneous routes. Suitable liquid compositions may be aqueous or non-aqueous, isotonic sterile injection solutions, and may contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Oral administration, parenteral administration, subcutaneous administration and intravenous administration are preferred methods of administration.

The dosage of the compounds of the present disclosure varies depending on a patient's age, weight, or symptoms, as well as the compound's potency or therapeutic efficacy, the dosing regimen and/or treatment time. Generally, suitable routes of administration may, for example, include oral, eyedrop, rectal, transmucosal, topical, or intestinal administration; parenteral delivery, including intramuscular, subcutaneous, intramedullary injections, as well as intrathecal, direct intraventricular, intravenous, intraperitoneal, intranasal, or intraocular injections. The compounds of the disclosure may be administered in an amount of 0.5 mg or 1 mg up to 500 mg, 1 g, or 2 g per dosage regimen. The dosage may be administered once per week, once per three days, once per two days, once per day, twice per day, three times per day, or more often. In alternative embodiments, in certain adults the compound can be continuously administered by intravenous administration for a period of time designated by a physician. Since the dosage is affected by various conditions, an amount less than or greater than the dosage ranges contemplated about may be implemented in certain cases. A physician can readily determine the appropriate dosage for a patient undergoing therapeutic treatment.

In certain embodiments, the present invention relates to a pharmaceutical composition, comprising at least one compound of formula (I), or a pharmaceutically acceptable salt or a stereoisomer thereof, and a pharmaceutically acceptable carrier or excipient for use in inhibiting FABP5 thereby inhibiting cancer cell proliferation associated with a deregulated lymphocyte receptor signaling pathway.

In a certain embodiments, the pharmaceutical composition further comprising at least one agent selected from an anticancer agent, a chemotherapy agent, and an antiproliferative compound for use in inhibiting FABP5 thereby inhibiting cancer cell proliferation associated with a deregulated lymphocyte receptor signaling pathway.

In certain embodiments, the pharmaceutical composition is useful for treating a patient with cancer associated with a deregulated lymphocyte receptor signaling pathway. In certain embodiments, the pharmaceutical composition is useful for treating a patient with B-cell cancer such as chronic lymphocytic leukemia (CLL), small lymphocytic lymphoma (SLL), diffuse large B-cell lymphoma (DLBCL), activated B-cell diffuse large B-cell lymphoma (ABC-DLBCL), germinal center diffuse large B-cell lymphoma (GCB DLBCL), primary mediastinal B-cell lymphoma (PMBL), non-Hodgkin lymphoma, Burkitt's lymphoma, follicular lymphoma, immunoblastic large cell lymphoma, precursor B-lymphoblastic lymphoma, precursor B-cell acute lymphoblastic leukemia, hairy cell leukemia, mantle cell lymphoma, B cell prolymphocytic leukaemia, lymphoplasmacytic lymphoma/Waldenstrom macroglobulinemia, splenic marginal zone lymphoma, plasma cell myeloma, plasmacytoma, extranodal marginal zone B cell lymphoma, nodal marginal zone B cell lymphoma, mediastinal (thymic) large B cell lymphoma, intravascular large B cell lymphoma, primary effusion lymphoma, or lymphomatoid granulomatosis.

In certain embodiments, the pharmaceutical composition is useful for treating a patient with T-cell cancer such as peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or a treatment-related T-cell lymphoma. In certain embodiments, T cell cancer is T-cell acute lymphoblastic leukaemia (T-ALL), peripheral T-cell lymphoma (PTCL), T-cell lymphoblastic lymphoma (T-CLL), cutaneous T-cell lymphoma (CTCL) or adult T-cell lymphoma (ATCL).

In certain embodiments, the pharmaceutical composition is useful for treating a patient with Hodgkin's lymphoma, Burkitt's lymphoma, non-Hodgkin's lymphoma, diffuse large B-cell lymphoma, or MALT lymphoma. In certain embodiments, the pharmaceutical composition is useful for treating a patient with diffuse large B-cell lymphoma.

The compositions and methods of the present disclosure may be utilized to treat a subject in need thereof. In certain embodiments, the subject is a mammal such as a human, or a non-human mammal. When administered to an animal, such as a human, the composition or the compound is preferably administered as a pharmaceutical composition comprising, for example, a compound of Formula (I) of the disclosure and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound of Formula (I) of the disclosure. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation of pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of Formula (I) of the disclosure. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of Formula (I) of the disclosure, with the carrier and, optionally, one or more accessory ingredients. In general, the formulations are prepared by uniformly and intimately bringing into association a compound of the present disclosure with liquid carriers, or finely divided solid carriers, or both, and then, if necessary, shaping the product.

Formulations of the disclosure suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. Compositions or compounds may also be administered as a bolus, electuary or paste.

To prepare solid dosage forms for oral administration (capsules including sprinkle capsules and gelatin capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present disclosure to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this disclosure. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference in its entirety. If desired, liquid ophthalmic formulations have properties similar to that of lacrimal fluids, aqueous humor or vitreous humor or are compatible with such fluids. A preferred route of administration is local administration (e.g., topical administration, such as eye drops, or administration via an implant).

A suppository also is contemplated as being within the scope of this disclosure.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this disclosure, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of Formula (I) of the disclosure. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the disclosure will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present disclosure, the active compound may be administered two or three times daily. In preferred embodiments, the active compound will be administered once daily.

The patient receiving this treatment is any animal in need, including primates, in particular humans, and other mammals such as equines, cattle, swine and sheep; and poultry and pets in general.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Methods of Administration

The compounds of the present disclosure may be used as single drugs (monotherapy) or conjointly with one or more other therapeutic agents (conjoint therapy). The compounds may be used by themselves, or, preferably, in a pharmaceutical composition in which the compound is mixed with one or more pharmaceutically acceptable materials.

In one embodiment, the present invention provides a method of inhibiting cancer cell proliferation with a deregulated lymphocyte receptor signaling pathway, further comprising contacting the cell with another therapeutic agent.

In one embodiment, the present invention provides a method of treating cancer in a subject having associated with a deregulated lymphocyte receptor signaling pathway further comprising administering to the subject another therapeutic agent.

In one embodiment, potential therapeutic agents to be combined with FABP5 inhibitor as described herein or a pharmaceutically acceptable salt, include but not restricted to biologic agents, Immune checkpoint modulators, epigenetic modulators, oncolytic viruses, and chemotherapeutic agents such as cytotoxic agents.

In certain embodiments, the FABP5 inhibitor of the present invention, i.e., a compound of formula (I) or a pharmaceutically acceptable salt or a stereoisomer thereof, can be administered either as a single drug or in combination with other therapeutic agents.

In one embodiment, the FABP5 inhibitor of the present invention is administered to the subject 1, 2, 3, 4, 5, 6, 8, 10, 12, 18, or 24 hours, 1, 2, 3, 4, 5, 6 or 7 days, 1, 2, 3 or 4 weeks, or any combination thereof prior to administration of other therapeutic agents to the subject.

In one embodiment, the therapeutic agent(s) is administered to the subject 1, 2, 3, 4, 5, 6, 8, 10, 12, 18, or 24 hours, 1, 2, 3, 4, 5, 6, or 7 days, 1, 2, 3, or 4 weeks, or any combination thereof prior to administration of FABP5 inhibitor of the present invention, to the subject. In another embodiment, the FABP5 inhibitor of the present invention and the therapeutic agent are administered sequentially.

As used herein, the term “oncolytic virus” refers to a virus capable of selectively replicating in dividing cells (e.g. a proliferative cell such as a cancer cell) with the purpose of slowing the growth and/or inducing the lysis of said dividing cell, either in vitro or in vivo, while showing no or minimal replication in non-dividing cells. Typically, an oncolytic virus contains a viral genome packaged into a viral particle (or virion) and is infectious (i.e. capable of infecting and entering into a host cell or subject).

In certain embodiments, the oncolytic virus is selected from the group consisting of reovirus, New Castle Disease virus (NDV), vesicular stomatitis virus (VSV), measles virus, influenza virus, Sinbis virus, adenovirus and poxvirus and herpes virus (HSV).

As used herein, an immune checkpoint modulator is an antagonist molecule that antagonizes the activity of PD-1, PD-L1 or CTLA-4. Exemplary immune checkpoint modulator include, but not limited to:
i. PD-1 inhibitors such as Pembrolizumab (formerly MK-3475 or lambrolizumab, Keytruda®), Nivolumab (Opdivo®), pidilizumab, AMP-224, AMP-514, PDR001, and cemiplimab.
ii. PD-L1ii. inhibitors such as Atezolizumab (Tecentriq®), Avelumab (Bavencio®), Durvalumab (Imfinzi®), BMS-936559, CK-301 (Iwai, et ak, Journal of Biomedical Science, (2017) 24:26)
iii. CTLA4 antagonists such as Ipilimumab, also known as MDX-010 or MDX-101, a human anti-CTLA4 antibody, preferably administered at a dose of about 10 mg/kg, and Tremelimumab a human anti-CTLA4 antibody, preferably administered at a dose of about 15 mg/kg. See also Sammartino, et a, Clinical Kidney Journal, 3 (2): 135-137 (2010), published online December 2009.

As used herein, the term “epigenetic modulators” refers to an agent that alters the epigenetic state (e.g., methylation state) of the DNA of a cell upon or after contact with or administration of such agent. In certain embodiments, the epigenetic modulators include a histone deacetylase (HDAC) inhibitor (HDACi). In certain embodiments the HDAC can be a Class I HDAC, a Class IIA HDAC, a Class IIB HDAC, a Class IV HDAC, or any combination thereof, or the HDAC can include a zinc-containing catalytic domain. In certain embodiments, the HDACi can bind to the zinc-containing catalytic domain of the HDAC. In certain embodiments, the HDACi can include a chemical moiety selected from the group consisting of a hydroxamic acid or a salt thereof, a cyclic tetrapeptide, a depsipeptide, a benzamide, an electrophilic ketone, an aliphatic acid or a salt thereof, or any combination thereof. For example, in certain embodiments, the HDACi is selected from e Vorinostat, Romidepsin, Chidamide, Panobinostat, Belinostat, Valproic acid or a salt thereof, Mocetinostat, bexinostat, Entinostat, Pracinostat, Resminostat, Givinostat, Quisinostat, Kevetrin, CUDC-101, AR-42, Tefinostat (CHR-2845), CHR-3996, 4SC-202, CG200745, ACY-1215, ACY-241, and any combination thereof, or any salt, crystal, amorphous structure, hydrate, derivative, metabolite, isomer, or prodrug thereof.

In certain embodiments, the epigenetic modulators include a DNA methyltransferase (DNMT) inhibitor (DNMTi). In certain embodiments the DNMT can be DNMT1, DNMT-3a, DNMT-3b, or any combination thereof. In certain embodiments, the DNMTi can be a nucleoside analog, an antisense oligonucleotide, a small molecule enzyme inhibitor, or any combination thereof. For example, in certain embodiments, the DNMTi is selected from azacytidine, decitabine, zebularine, SGI-110, epigallocatechin gallate, MG98, RG108, procainamide, hydralazine and any combination thereof, or any salt, crystal, amorphous structure, hydrate, derivative, metabolite, isomer, or prodrug thereof.

In one embodiment, chemotherapeutic agent are chemical compounds useful in the treatment of cancer. In one embodiment, compounds of the present invention, or a pharmaceutically acceptable composition thereof, are administered in combination with chemotherapeutic agent which includes erlotinib (TARCEVA®, Genentech/OSI Pharm.), bortezomib (VELCADE®, Millennium Pharm.), disulfiram, epigallocatechin gallate, salinosporamide A, carfilzomib, 17-AAG(geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5a-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancrati statin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin [symbol] and calicheamicin coll (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, 111.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

In one embodiment, biologics agents include antibodies such as alemtuzumab (Campath), bevacizumab (A VASTEST®, Genentech); cetuximab (ERBITUX®, Imclone); panitumumab (VECTIBIX®, Amgen), rituximab (RITUXAN®, Genentech/Biogen Idee), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgGi λ antibody genetically modified to recognize interleukin-12 p40 protein.

### Definitions

Unless defined otherwise, all technical and scientific terms used herein have the same meaning as is commonly understood by one of skill in art to which the subject matter herein belongs. As used in the specification and the appended claims, unless specified to the contrary, the following terms have the meaning indicated in order to facilitate the understanding of the present invention.

The singular forms “a”, “an” and “the” encompass plural references unless the context clearly indicates otherwise.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to an event or circumstance in which the said alkyl group may be substituted as well as the event or circumstance where the alkyl group is not substituted. In one embodiment, the expression “optionally substituted” can be interchangeably termed as “substituted or unsubtituted”.

The term “substituted” refers to moieties having substituents replacing hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, an oxo, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heteroaryl, a heterocycloalkyl, an aralkyl, or an aromatic or heteroaromatic moiety. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

As used herein, the term “alkyl” refers to saturated aliphatic groups, including but not limited to C₁-C₁₀ straight-chain alkyl groups or C₃-C₁₀ branched-chain alkyl groups. Preferably, the “alkyl” group refers to C₁-C₆ straight-chain alkyl groups or C₃-C₆ branched-chain alkyl groups. In one embodiment, the “alkyl” group refers to C₁-C₄ straight-chain alkyl groups or C₃-C₈ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl and 4-octyl. The “alkyl” group may be optionally substituted.

As used herein, the term “heteroalkyl” refers to a straight- or branched-chain alkyl group in which one or more of carbon atoms have been replaced by a heteroatom selected from S, O, P and N; wherein the ‘alkyl’ group is as defined above. Exemplary heteroalkyl' s include alkyl ethers, secondary and tertiary alkyl amines, amides, alkyl sulfides and alkyl disulfides. The group, may be a terminal group or a bridging group.

As used herein, the term “alkenyl” refers to a carbon chain which contains at least one carbon-carbon double bond, and which may be linear or branched or combinations thereof. Examples of “alkenyl” include, but not limited to, vinyl, allyl, isopropenyl, pentenyl, hexenyl, heptenyl, 1-propenyl, 2-butenyl and 2-methyl-2-butenyl.

By analogy, the expression “alkenylene” refers to a divalent “alkenyl” radical as above defined.

As used herein, the term “alkynyl” refers to straight or branched carbon chains with one or more triple bonds wherein the number atoms is in the range 2 to 6.

By analogy, the expression “alkynylene” refers to a divalent “alkynyl” radical as above defined.

As used herein, the term “haloalkyl” means alkyl substituted with one or more halogen atoms, wherein the halo and alkyl groups are as defined above. The term “halo” is used herein interchangeably with the term “halogen” means F, Cl, Br or I. In one embodiment, haloalkyl contains (C₁-C₆)alkyl and preferably (C₁-C₄)alkyl. Examples of “haloalkyl” include but are not limited to fluoromethyl, difluoromethyl, chloromethyl, trifluoromethyl and 2,2,2-trifluoroethyl.

As used herein, the term “hydroxy” or “hydroxyl” alone or in combination with other term(s) means —OH.

As used herein the term “hydroxyalkyl” or “hydroxylalkyl” means alkyl substituted with one or more hydroxyl groups, wherein the alkyl groups are as defined above. In one embodiment, hydroxyalkyl contains (C₁-C₆)alkyl and preferably (C₁-C₄)alkyl. Examples of “hydroxyalkyl” include but are not limited to, hydroxymethyl, hydroxyethyl, hydroxypropyl and propan-2-ol.

The term “ester”, as used herein, refers to a group —C(O)SR [symbol] wherein R [symbol] represents a hydrocarbyl group.

The term “carboxy” or “carboxylic acid”, as used herein, refers to a group represented by the formula —CO₂H.

The term “thioester”, as used herein, refers to a group —C(O)SR [symbol] or —SC(O)R [symbol] wherein R [symbol] represents a hydrocarbyl.

As used herein, the term “hydrocarbyl” is a group having a carbon atom directly attached to the remaining part of the molecule having hydrocarbon character.

As used herein, the term “oxo” refers to ═O group.

As used herein, the term “alkoxy” refers to the group —O-alkyl, where alkyl groups are as defined above. Exemplary C₁-C₁₀ alkoxy group include but are not limited to methoxy, ethoxy, n-propoxy, n-butoxy or t-butoxy. In one embodiment, the “alkoxy” group refers to C₁-C₆ alkoxy groups. In one embodiment, the “alkoxy” group refers to C₁-C₄ alkoxy groups. An alkoxy group can be optionally substituted with one or more suitable groups.

As used herein, the term “alkoxyaryl” refers to the group —O-alkyl, which is attached aryl group, where alkyl and aryl groups are as defined in this specification.

As used herein, the term “cyano” refers to —CN group.

As used herein, “amino” refers to an —NH₂ group.

As used herein, “amido” refers to an —CONH₂ group.

As used herein, “alkylamino” or “cycloalkylamino”, refer to an —NH₂ group, wherein nitrogen atom of said group being attached to one or two alkyl or cycloalkyl groups respectively. Representative examples of an “alkylamino” and “cycloalkylamino” groups include, but are not limited to —NHCH₃ and —NH-cyclopropyl. The term “alkylamino” also includes dialkylamino (e.g., —N(CH₃)₂) groups.

“Aminoalkyl” refers to an alkyl group, as defined above, wherein one or more of the alkyl group's hydrogen atom has been replaced with an amino group as defined above. Representative examples of an aminoalkyl group include, but are not limited to —CH₂NH₂, —CH₂CH₂NH₂, —CH(CH₃)NH₂, —CH₂CH(CH₃)NH₂. An aminoalkyl group can be unsubstituted or substituted with one or more suitable groups.

As used herein the term “cycloalkyl” alone or in combination with other term(s) means —C₃-C₁₀ saturated cyclic hydrocarbon ring. A cycloalkyl may be a single ring, which typically contains from 3 to 7 carbon ring atoms. Examples of single-ring cycloalkyls include but are not limited to cyclopropyl, cyclobutyl, cyclopentyl, cyclohexyl and cycloheptyl. A cycloalkyl may alternatively be polycyclic or contain more than one ring. Examples of polycyclic cycloalkyls include bridged, fused and spirocyclic carbocyclyls.

As used herein, the term “heterocycloalkyl” refers to a non-aromatic, saturated or partially saturated, monocyclic or polycyclic ring system of 3 to 15 member having at least one heteroatom or heterogroup selected from O, N, S, S(O), S(O)₂, NH or C(O) with the remaining ring atoms being independently selected from the group consisting of carbon, oxygen, nitrogen, and sulfur. The term “heterocycloalkyl” also refers to the bridged bicyclic ring system having at least one heteroatom or heterogroup selected from O, N, S, S(O), S(O)₂, NH or C(O). Examples of “heterocycloalkyl” include, but are not limited to azetidinyl, oxetanyl, imidazolidinyl, pyrrolidinyl, oxazolidinyl, thiazolidinyl, pyrazolidinyl, tetrahydrofuranyl, piperidinyl, piperazinyl, tetrahydropyranyl, morpholinyl, thiomorpholinyl, 1,4-dioxanyl, dioxidothiomorpholinyl, oxapiperazinyl, oxapiperidinyl, tetrahydrofuryl, tetrahydropyranyl, tetrahydrothiophenyl, dihydropyranyl, indolinyl, indolinylmethyl, aza-bicyclooctanyl, azocinyl, chromanyl, xanthenyl and N-oxides thereof. Attachment of a heterocycloalkyl substituent can occur via either a carbon atom or a heteroatom. A heterocycloalkyl group can be optionally substituted with one or more suitable groups by one or more aforesaid groups. Preferably “heterocycloalkyl” refers to 5- to 6-membered ring selected from the group consisting of imidazolidinyl, pyrrolidinyl, oxazolidinyl, thiazolidinyl, pyrazolidinyl, tetrahydrofuranyl, piperidinyl, piperazinyl, tetrahydropyranyl, morpholinyl, thiomorpholinyl, 1,4-dioxanyl and N-oxides thereof. More preferably, “heterocycloalkyl” includes azetidinyl, pyrrolidinyl, morpholinyl and piperidinyl. All heterocycloalkyl are optionally substituted by one or more aforesaid groups.

As used herein, the term “(heterocycloalkyl)alkyl” refers to the group alkyl, attached heterocycloalkyl group, where ‘alkyl’ and heterocycloalkyl' groups are as defined in this specification.

As used herein, the term “heteroaryl” refers to an aromatic heterocyclic ring system containing 5 to 20 ring atoms, suitably 5 to 10 ring atoms, which may be a single ring (monocyclic) or multiple rings (bicyclic, tricyclic or polycyclic) fused together or linked covalently. Preferably, “heteroaryl” is a 5- to 6-membered ring. The rings may contain from 1 to 4 heteroatoms selected from N, O and S, wherein the N or S atom is optionally oxidized or the N atom is optionally quarternized. Any suitable ring position of the heteroaryl moiety may be covalently linked to the defined chemical structure.

Examples of heteroaryl include, but are not limited to: furanyl, thienyl, pyrrolyl, pyrazolyl, imidazolyl, oxazolyl, cinnolinyl, isoxazolyl, thiazolyl, isothiazolyl, 1H-tetrazolyl, oxadiazolyl, triazolyl, pyridyl, pyrimidinyl, pyrazinyl, pyridazinyl, benzoxazolyl, benzisoxazolyl, benzothiazolyl, benzofuranyl, benzothienyl, benzotriazinyl, phthalazinyl, thianthrene, dibenzofuranyl, dibenzothienyl, benzimidazolyl, indolyl, isoindolyl, indazolyl, quinolinyl, isoquinolinyl, quinazolinyl, quinoxalinyl, purinyl, pteridinyl, 9H-carbazolyl, a-carboline, indolizinyl, benzoisothiazolyl, benzoxazolyl, pyrrolopyridyl, pyrazolopyrimidyl, furopyridinyl, purinyl, benzothiadiazolyl, benzooxadiazolyl, benzotriazolyl, benzotriadiazolyl, carbazolyl, dibenzothienyl, acridinyl and the like. Preferably “heteroaryl” refers to 5- to 6-membered ring selected from the group consisting of furanyl, thienyl, pyrrolyl, pyrazolyl, imidazolyl, oxazolyl, cinnolinyl, isoxazolyl, thiazolyl, isothiazolyl, 1H-tetrazolyl, oxadiazolyl, triazolyl, pyridyl, pyrimidinyl, pyrazinyl and pyridazinyl. More preferably, pyrazolyl, pyridyl, oxazolyl and furanyl. All heteroaryls are optionally substituted by one or more aforesaid groups.

As used herein, the term “aryl” is optionally substituted monocyclic, bicyclic or polycyclic aromatic hydrocarbon ring system of about 6 to 14 carbon atoms. In one embodiment, “aryl” refers to C₆-C₁₀ aryl group. Examples of a C₆-C₁₄ aryl group include, but are not limited to phenyl, naphthyl, biphenyl, anthryl, fluorenyl, indanyl, biphenylenyl and acenaphthyl. Aryl group can be unsubstituted or substituted with one or more suitable groups.

As used herein, the term “aryloxy” refers to the group —O-aryl, where aryl groups are as defined above. Exemplary “aryloxy” group include but are not limited to phenoxy or napthyl-oxy.

The term “acyl” refers to a group R—CO— wherein R is an optionally substituted alkyl group 20 defined above. Examples of ‘acyl’ groups are, but not limited to, CH₃CO—, CH₃CH₂CO—, CH₃CH₂CH₂CO— or (CH₃)₂CHCO—.

As used herein, the terms “B-cell cancer” and “T-cell cancer” refer to a group of heterogeneous cancers of the white blood cells known as B-lymphocytes or B-cells (bone marrow-derived cells) and T-lymphocytes or T-cells (thymus-derived cells), respectively. Broad examples of B-cell cancer and T-cell cancer include leukemias (located in the blood) and lymphomas (located in the lymph nodes) such as B-cell leukemias, B-cell lymphomas, T-cell leukemias and B-cell lymphoma.

As used herein, the term “compound(s)” comprises the compound(s) disclosed in the present invention.

As used herein, the term “comprise” or “comprising” is generally used in the sense of include, that is to say permitting the presence of one or more features or components.

As used herein, the term “or” means “and/or” unless stated otherwise.

As used herein, the term “including” as well as other forms, such as “include”, “includes” and “included” is not limiting.

As used herein, the term “composition” is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

As used herein, the term “pharmaceutical composition” refers to a composition(s) containing a therapeutically effective amount of at least one compound of formula (I) or a pharmaceutically acceptable salt or a stereoisomer thereof; and a pharmaceutically acceptable carrier.

The pharmaceutical composition(s) usually contain(s) about 1% to 99%, for example, about 5% to 75%, or from about 10% to about 30% by weight of the compound of formula (I) or pharmaceutically acceptable salts thereof. The amount of the compound of formula (I) or pharmaceutically acceptable salts thereof in the pharmaceutical composition(s) can range from about 1 mg to about 1000 mg or from about 2.5 mg to about 500 mg or from about 5 mg to about 250 mg or in any range falling within the broader range of 1 mg to 1000 mg or higher or lower than the afore mentioned range.

As used herein, the term “genetic alterations” refers to any change in the genome leading to a change in DNA sequence, mRNA sequence, protein sequence, changes in gene expression (either mRNA or protein abundance), or combinations thereof. Genentic alterations includes, but not limited to, deleterious mutations (e.g., mutations that reduce or abolish either gene function or gene expression), loss of function mutations, gain of function mutations and others. Genetic alterations includes insertions of viral genetic material into the genome of infected host cells (e.g., human papillomavirus). Genetic alterations also includes microsatellites or other repetitive tracts of DNA (e.g., short tandem repeats or simple sequence repeats).

As used herein, “loss of function” (LOF) mutation refers to a mutation or allele of a gene, the result of which is that the gene product (such as the encoded protein) has less than normal or no function in a cell or organism (including a human cell or human being). When the allele has a complete loss of function (null allele) it is often called an amorphic mutation. Phenotypes associated with loss of function mutations are often recessive.

As used herein, the term “overexpression” when referring to a gene (e.g., an oncogenic driver gene), refers to any increase in mRNA, protein, or combinations thereof corresponding to a gene compared to normal level.

As used herein, the term “treat”, “treating” and “treatment” refer to a method of alleviating or abrogating a disease and/or its attendant symptoms.

As used herein, the term “prevent”, “preventing” and “prevention” refer to a method of preventing the onset of a disease and/or its attendant symptoms or barring a subject from acquiring a disease. As used herein, “prevent”, “preventing” and “prevention” also include delaying the onset of a disease and/or its attendant symptoms and reducing a subject's risk of acquiring a disease.

As used herein, the term “subject” that may be interchangeable with ‘patient’, refers to an animal, preferably a mammal, and most preferably a human.

As used herein, the term, “therapeutically effective amount” refers to an amount of a compound of formula (I) or a pharmaceutically acceptable salt or a stereoisomer thereof; or a composition comprising the compound of formula (I) or a pharmaceutically acceptable salt or a stereoisomer thereof, effective in producing the desired therapeutic or pharmacological response in a particular patient suffering from a diseases or disorder described herein, in particular their use in diseases or disorder associated with cancer. Particularly, the term “therapeutically effective amount” includes the amount of the compound of formula (I) or a pharmaceutically acceptable salt or a stereoisomer thereof, when administered, that induces a positive modification in the disease or disorder to be treated or is sufficient to prevent development of, or alleviate to some extent, one or more of the symptoms associated with the disease or disorder being treated in a subject. In respect of the therapeutic amount of the compound, the amount of the compound used for the treatment of a subject is low enough to avoid undue or severe side effects, within the scope of sound medical judgment can also be considered. The therapeutically effective amount of the compound or composition will be varied with the particular condition being treated, the severity of the condition being treated or prevented, the duration of the treatment, the nature of concurrent therapy, the age and physical condition of the end user, the specific compound or composition employed the particular pharmaceutically acceptable carrier utilized.

The term “pharmaceutically acceptable salt” refers to a product obtained by reaction of the compound of the present invention with a suitable acid or a base. Pharmaceutically acceptable salts of the compounds of this invention include those derived from suitable inorganic bases such as Li, Na, K, Ca, Mg, Fe, Cu, Al, Zn and Mn salts; Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain compounds of the invention (compound of formula (I)) can form pharmaceutically acceptable salts with various organic bases such as lysine, arginine, guanidine, diethanolamine or metformin. Suitable base salts include, but are not limited to, aluminum, calcium, lithium, magnesium, potassium, sodium or zinc salts.

The present invention also provides methods for formulating the disclosed compounds as for pharmaceutical administration.

In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

The term “stereoisomers” refers to any enantiomers, diastereoisomers or geometrical isomers of the compounds of formula (I), wherever they are chiral or when they bear one or more double bonds. When the compounds of the formula (I) and related formulae are chiral, they can exist in racemic or in optically active enantiomeric form. It should be understood that the invention encompasses all stereochemical isomeric forms, including diastereomeric, enantiomeric and epimeric forms, as well as d-Isomers and l-Isomers and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds of the present invention may exist as geometric Isomers. The present invention includes all cis, trans, syn, anti, entgegen (E) and zusammen (Z) Isomers as well as the appropriate mixtures thereof.

The compounds of the present invention may be used as single drug or as a pharmaceutical composition in which the compound is mixed with various pharmacologically acceptable materials.

The compounds of the invention are typically administered in the form of a pharmaceutical composition. Such compositions can be prepared using procedures well known in the pharmaceutical art and comprise at least one compound of the invention. The pharmaceutical composition of the present patent application comprises one or more compounds described herein and one or more pharmaceutically acceptable excipients. Typically, the pharmaceutically acceptable excipients are approved by regulatory authorities or are generally regarded as safe for human or animal use. The pharmaceutically acceptable excipients include, but are not limited to, carriers, diluents, glidants and lubricants, preservatives, buffering agents, chelating agents, polymers, gelling agents, viscosifying agents and solvents.

The pharmaceutical composition can be administered by oral, parenteral or inhalation routes. Examples of the parenteral administration include administration by injection, percutaneous, transmucosal, transnasal and transpulmonary administrations.

Examples of suitable carriers include, but are not limited to, water, salt solutions, alcohols, polyethylene glycols, peanut oil, olive oil, gelatin, lactose, terra alba, sucrose, dextrin, magnesium carbonate, sugar, amylose, magnesium stearate, talc, gelatin, agar, pectin, acacia, stearic acid, lower alkyl ethers of cellulose, silicic acid, fatty acids, fatty acid amines, fatty acid monoglycerides and diglycerides, fatty acid esters and polyoxyethylene.

The pharmaceutical composition may also include one or more pharmaceutically acceptable auxiliary agents, wetting agents, suspending agents, preserving agents, buffers, sweetening agents, flavouring agents, colorants or any combination of the foregoing.

The pharmaceutical compositions may be in conventional forms, for example, tablets, capsules, solutions, suspensions, injectables or products for topical application. Further, the pharmaceutical composition of the present invention may be formulated so as to provide desired release profile.

Administration of the compounds of the invention, in pure form or in an appropriate pharmaceutical composition, can be carried out using any of the accepted routes of administration of pharmaceutical compositions. The route of administration may be any route which effectively transports the active compound of the patent application to the appropriate or desired site of action. Suitable routes of administration include, but are not limited to, oral, nasal, buccal, dermal, intradermal, transdermal, parenteral, rectal, subcutaneous, intravenous, intraurethral, intramuscular or topical.

Solid oral formulations include, but are not limited to, tablets, capsules (soft or hard gelatin), dragees (containing the active ingredient in powder or pellet form), troches and lozenges.

Liquid formulations include, but are not limited to, syrups, emulsions, and sterile injectable liquids, such as suspensions or solutions.

Topical dosage forms of the compounds include ointments, pastes, creams, lotions, powders, solutions, eye or ear drops, impregnated dressings, and may contain appropriate conventional additives such as preservatives, solvents to assist drug penetration.

The pharmaceutical compositions of the present patent application may be prepared by conventional techniques known in literature.

Suitable doses of the compounds for use in treating the diseases or disorders described herein can be determined by those skilled in the relevant art. Therapeutic doses are generally identified through a dose ranging study in humans based on preliminary evidence derived from the animal studies. Doses must be sufficient to result in a desired therapeutic benefit without causing unwanted side effects. Mode of administration, dosage forms, and suitable pharmaceutical excipients can also be well used and adjusted by those skilled in the art. All changes and modifications are envisioned within the scope of the present patent application.

The synthetic procedures for the preparation of compounds of this disclosure were described in WO2019142126 A1 which is incorporated herein in its entirety.

### Example—1: Determination of Anti Proliferative Activity in Haematological Cancer Cell Lines

OCI-LY3 cells (DSMZ ACC 761), OCI-LY10 (DSMZ ACC 722) were plated in 96 well flat black clear bottom plates (Corning, Cat. No 3904) using complete IMDM complete media. Pfeiffer (ATCC CRL-2632), TMD8 (CVCL_A442), HBL-1 (CVCL_4213), DOHH2 (DSMZ ACC 47), CCRF-CEM [ATCC CCL-119], CUTLL-1 (CVCL_4966) and NCI-H929 [H929] ATCC CRL-9068 were plated in 96 well flat black clear bottom plates (Corning, Cat. No 3904) using RPMI-1640 complete media.

After 24 hours, selected compound of the present invention was added to cells from 10 mM stocks made in DMSO (Sigma Cat no. D2650). CTG reading was taken on the day of compound addition which was labelled as Day 0 reading. Each concentration of compound was tested in triplicate with DMSO concentration not exceeding a final percentage of 0.1 in the cells. After 3 days (72 hours) of incubation assay terminated using 100 μl of CellTiter Glo® reagent (Promega, Cat. no G7572). CellTiter Glo® Luminescent reagent determines the number of viable cells based on quantitation of ATP present which is an indicator of cell number and metabolically activity. Luminescence readings were taken in Victor-3 instrument. The data was analysed using graph pad prism software. The results are shown in Table-II. Positive control (100% survival)=Cells in complete media with 0.3% DMSO; Negative control/blank (0% survival)=Media alone containing 0.1% DMSO.

TABLE-II
Anti proliferative activity of compounds of present
invention in hematological cancer cell lines

| | | IC₅₀ (μM) | |
|---|---|---|---|
| Cell Line | Origin | Cpd 23 | Ibrutinib |
| OCI-LY3 | ABC-DLBCL | 0.02 | >10 |
| OCI-LY10 | | 0.01 | 0.001 |
| HBL-1 | | 0.35 | 0.72 |
| TMD8* (wt btk) | | 0.01 | <0.001 |
| Pfeiffer | GCB-DLBCL | 0.87 | 0.16 |
| DOHH-2 | Follicular lymphoma | 0.02 | 0.03 |
| CCRF-CEM | T-cell acute lymphoblastic | 0.28 | >3 |
| CUTLL-1 | leukemia | 0.05 | >3 |
| H929 | Multiple myeloma | 0.05 | >10 |

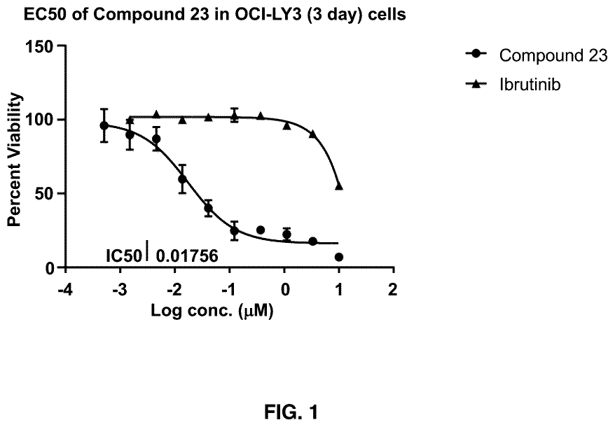

As shown above (Table-II), potency of compound of present invention, FABP5 inhibitor, matched that of BTK inhibitor ibrutinib in most of the cancer cell lines supporting the potential for the FABP5 inhibitors in cancer indications where BTK inhibitor ibrutinib is effective. Interestingly, in selected cell lines including OCI-LY3 (ABC-DLBCL), CCRF-CEM, CUTLL-1 (both T-cell acute lymphoblstic leukemia) and H929 (multiple myeloma), Cpd 23, FABP5 inhibitor, showed potent anti-proliferative activity while BTK inhibitor ibrutinib was not active. Further, Cpd 23 showed potent anti-proliferative activity in OCI-LY3 (ABC-DLBCL) (FIG. 1), a cell line that is resistant to BTK inhibitor because of the presence of an activating mutation in CARD11 (a signaling intermediate downstream of BTK). Potent activity of FABP5 inhibitor in these cell lines that are intrinsically resistant to ibrutinib supports that FABP5 inhibitors can be used for the treatment of BTK inhibitor-resistant cancers.

### Example—2: Inhibition of Cellular MALT1 Activity

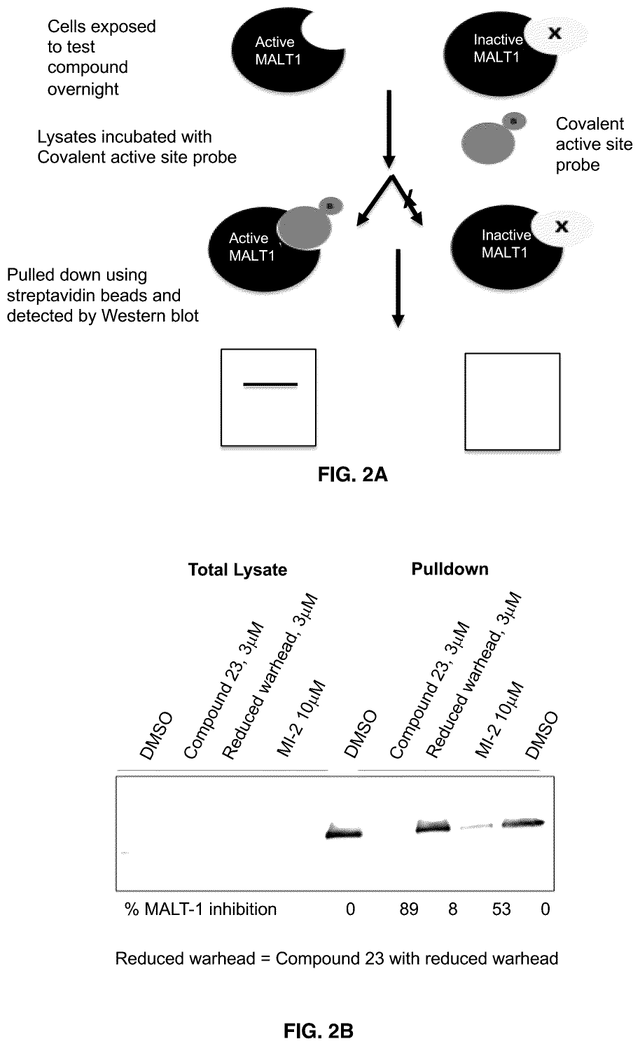

OCI-LY3 cells were incubated with Compound 23 at indicated concentrations (MI-2, an MALT1 inhibitor was used as reference) overnight after which lysates were incubated with biotinylated active site probe (peptide) that can bind MALT1 in a covalent manner. Following this, the lysate was pulled down using streptavidin conjugated beads (Millipore cat #S1638) followed by detection of streptavidin label (R&D systems cat no Dy998) using Western blot (FIG. 2)

### Example—3: Stabilization of MALT1 Substrates

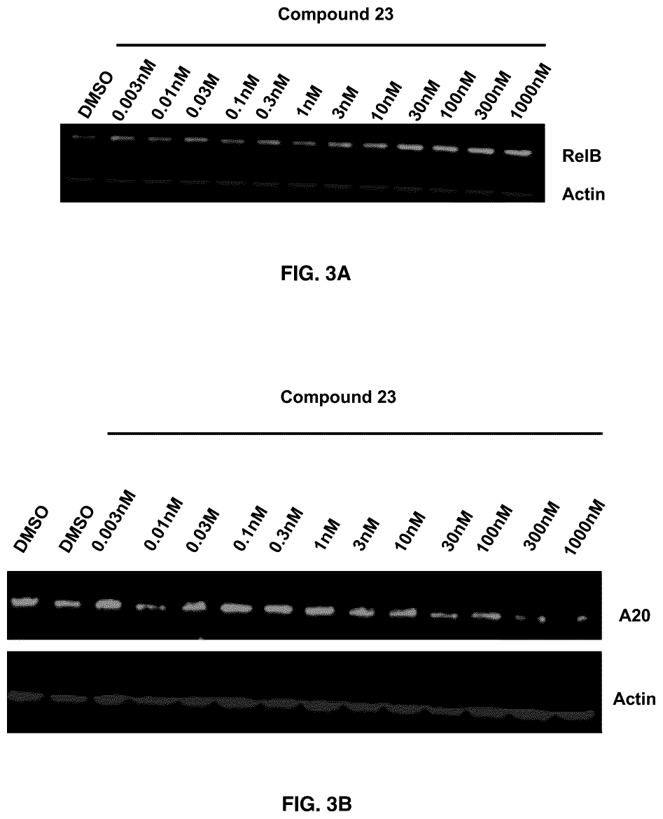

OCI-LY3 cells (DSMZ ACC 761) were seeded in a 6-well plate with complete IMDM media and incubated with a range of concentrations of the compound for 40 hours. This was followed by Western blot with these cell lysates using antibodies to RelB (CST cat. No. 4922), A20 (Cell Signaling technologies 4625S) and beta actin (Sc-69879). Band intensities of RelB and β-Actin were estimated from the raw data image file using Image studio software and exported to an excel sheet. Blots were dried with tissue paper and scanned LICOR Odyssey™ infrared scanner in the 800 and 680 channels (FIGS. 3A & 3B).

### Example—4: Inhibition of Cytokine Release

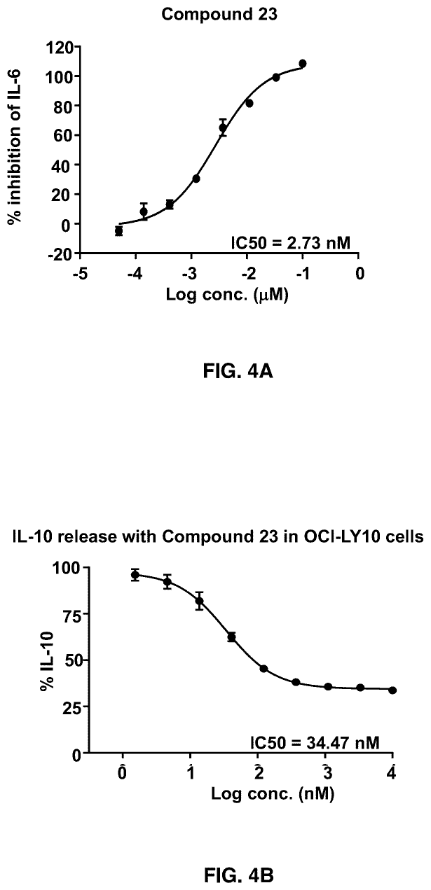

For evaluation of the impact of Compound 23 on IL-6 secretion, OCI-LY3 cells (DSMZ ACC 761) were seeded in 96-well plates (Corning cat no. CLS3596) with complete IMDM media and incubated with a range of concentrations of the compound for 19 hours. After 19 hours of incubation, culture supernatant was collected into fresh 96 well plate by centrifuging the plate which was then stored at −70±10° C. until used for ELISA. Supernatants were processed for human IL-6 measurement. ELISA which was performed by following manufacturers protocol (R&D System DY206). Percentage IL-6 inhibition was calculated as listed below and was plotted against respective concentration of the test item using Graph Pad Prism, Version 7.03 software to calculate IC₅₀ values. The results are shown in FIG. 4A.

For evaluation of the impact of compound 23 on IL-10 secretion, OCI-Ly10 cells (DSMZ ACC 722) were seeded in 96-well plates (Corning cat no. CLS3596) with complete IMDM media and incubated with a range of concentrations of the compound for 16 hours. After 16 hours of incubation, culture supernatant was collected into fresh 96 well plate by centrifuging the plate which was then stored at −70±10° C. until used for ELISA. Supernatants were processed for human IL-10 measurement. ELISA which was performed by following manufacturers protocol (R&D System DY217B). Standard graph was plotted using known concentrations of standards and respective absorbance values obtained after ELISA. IL-10 concentration in pg/ml was plotted against respective concentration of the test item using Graph Pad Prism, Version 7.03 software to calculate IC₅₀ values. Percentage IL-6 inhibition was calculated as listed below and was plotted against respective concentration of the test item using Graph Pad Prism, Version 7.03 software to calculate IC₅₀ values. The results are shown in FIG. 4B.

### Example—5: Inhibition of NFAT and NF-kB

### NFAT Reporter Assay

Jurkat cells were seeded in RPMI complete media in 96-well flat bottom white plates (Corning #3912) and incubated with compound 23 for 16 hours after which they were stimulated with PMA (1 μM) and Ionomycin (4 μM). After six hours, NFAT Reporter analysis was done according to manufacturer protocol (BPS Biosciences #60621).

### NF-kB Reporter Assay

Jurkat cells were seeded in RPMI complete media in 96-well flat bottom white plates (Corning #3912) and incubated with compound 23 for 1 hour after which they were stimulated with PMA (1 μM) and Ionomycin (4 μM). After six hours, NF-kB Reporter analysis was done according to manufacturer protocol (BPS Biosciences #60651).

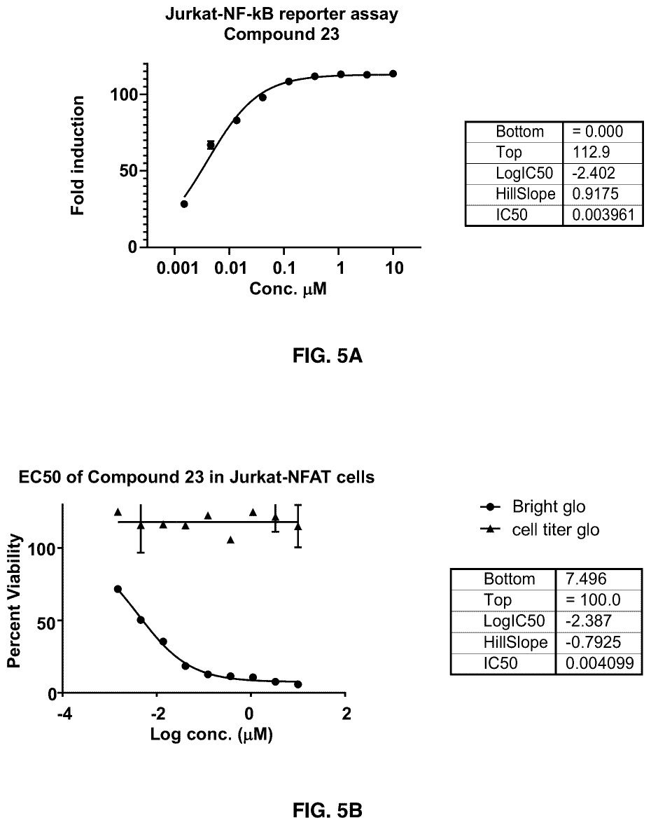

The results are shown in FIG. 5A and FIG. 5B.

### Example—6: In-vivo Tumour Growth Inhibition in Human DLBCL Tumour Model

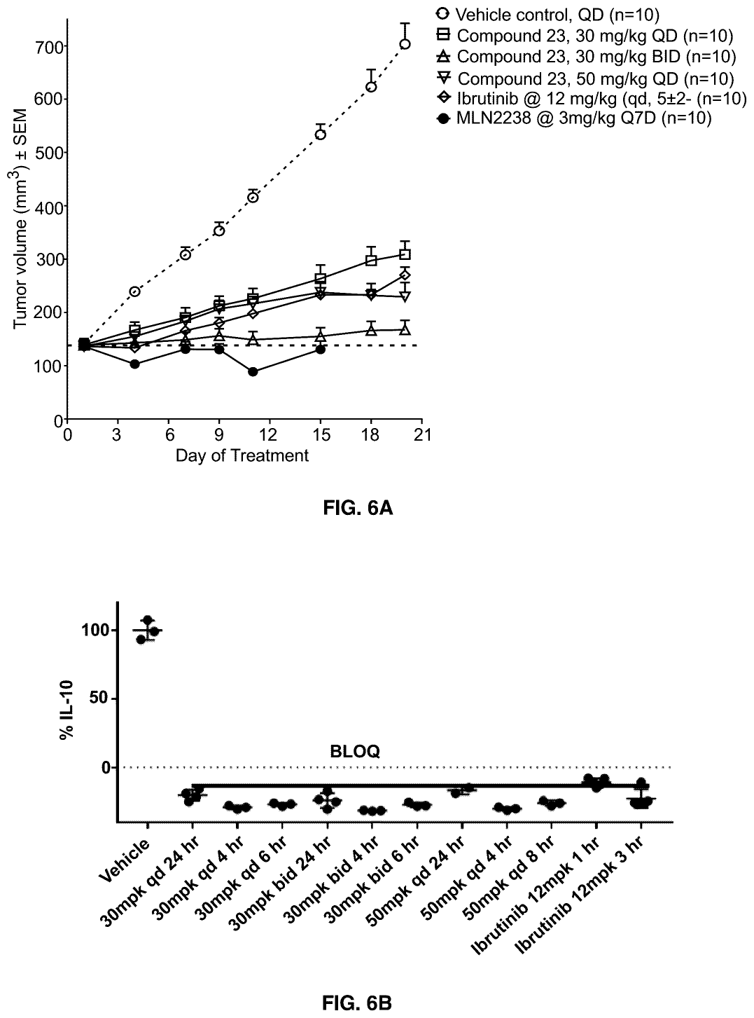

To evaluate anti-tumor activity of compound 23 in OCI-LY10 human DLBCL model in female NOD-SCID mice, dosing with vehicle, compound 23 and Ibrutinib was initiated. Treatments were administered oral route of administration at doses of 30 to 50 mpk qd as well as 30 mpk bid for 21 days. Overall efficacy and tolerability were evaluated based on tumor volume and body weight changes observed during the treatment period. On treatment day 21, animals from all the treatment groups were sacrificed at 4, 6 and 24 hours after last dose administration. Cytokine (human IL-10) measurement was also performed in serum and tumor samples using ELISA kit (R&D systems #DY217B) as per manufacturer instructions. The results are shown in FIG. 6A, FIG. 6B and FIG. 6C.

### Example—7: Cellular Thermal Shift Assay for FABP5 in OCI-Ly10 Cells

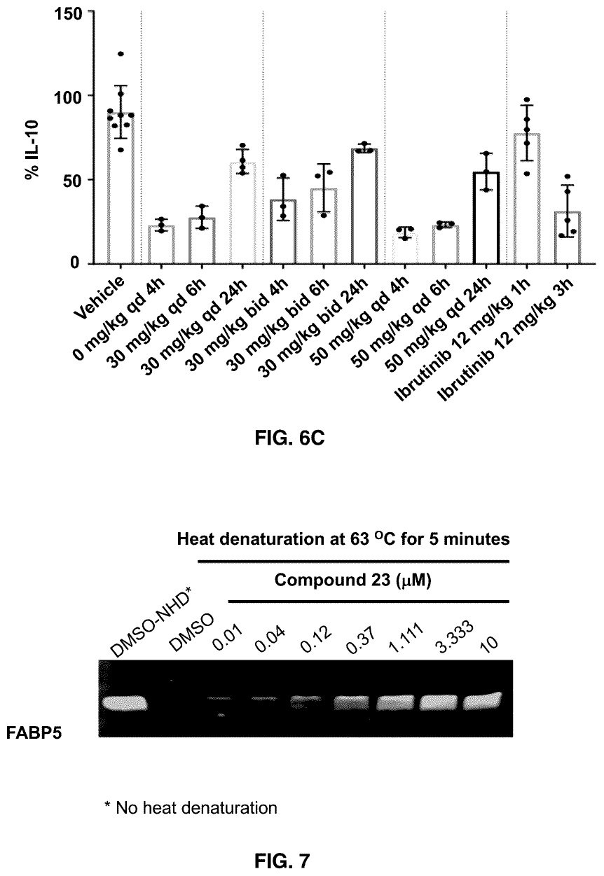

OCI-Ly10 cells (1.2 million cells/well) were seeded in 12 well plates and treated with serial dilutions of compound 23 for 24 hours (0.1% DMSO was included as control). The cells for each treatment were harvested, resuspended in 50 μL of PBS and subjected to heat denaturation (62° C. for 5 min) in PCR tubes using thermal cycler. The PCR tubes were transferred to ice and 10 μL CST lysis buffer solution with PMSF was added to all tubes including non heat denatured (NHD) control tube, mixed well and contents were transferred to 1.5 mL tubes. The tubes were incubated on ice for 30 min, sonicated for 10 seconds and centrifuged at 15000 rpm for 15 min. The supernatants were transferred to 1.5 mL tubes and samples for Western blot were prepared adding 10 μL Protein loading dye. The samples were boiled at 95° C. for 5-8 min, resolved using 15% SDS PAGE gel (50 V) and transferred to PVDF membrane (35 V for 70 min). The PVDF membrane was blocked using LICOR blocking buffer for 1 hour at RT and incubated with FABP5 primary antibody (Sino Biologicals #12581-T5; 1:2000 dilution in blocking buffer) overnight at 4° C. The membrane was washed (3×) with TBST and incubated with IRDYE-800 anti-rabbit antibody (1:10000 dilution in blocking buffer) for 1 hour at RT. The membrane was washed (3×) with TBST and image was acquired using Licor scanner. The results are shown in FIG. 7.

### INNCORPORATION BY REFERENCE

All publications and patents mentioned herein are hereby incorporated by reference in their entirety as if each individual publication or patent were specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of inhibiting cancer cell proliferation associated with a deregulated lymphocyte receptor signaling pathway, comprising contacting the cancer cell with a fatty acid-binding protein 5 (FABP5) inhibitor.

2. The method of claim 1, wherein the deregulated lymphocyte receptor signaling is a deregulated B-cell receptor signaling (BCR) or a deregulated T-cell receptor signaling (TCR).

3. The method of claim 1, wherein the deregulated lymphocyte receptor signaling is the deregulated BCR, which is associated with the genetic alterations in BCR signaling mediator; wherein the BCR signaling mediator is CD79, BTK, MALT1, BCL-10, BCL2, TRAF2, TRAF6, TAK1, CARD9, CARD10 (or CARMA3), CARD11 (or CARMA1), CARD14 (or CARMA2), TAB1, TAB2, TAB3, TAK1, IKKα, IKKβ, IKKγ, AP11, AP12, AP13, AP14 or A20.

4-5. (canceled)

6. The method of claim 1, wherein the deregulated B-cell receptor (BCR) signaling pathway is further associated with the genetic alterations in IKBKB, NFKBIA, NFKBIE, TNFAIP3, TRAF3, BIRC3, MAP3K14, IKK complex, CBM complex, NF-ϰB target genes or MAPK target genes.

7. The method of claim 2, wherein the deregulated B-cell receptor (BCR) signaling pathway is further associated with the genetic alterations in TCF3 genes or ID3 genes.

8. The method of claim 1, wherein the deregulated lymphocyte receptor signaling is the deregulated TCR, which is associated with the genetic alterations in TCR signaling mediator; wherein the TCR signaling mediator is FYN, ITK, SYK, PLC-gamma, MALT1, BCL-10, BCL2, TRAF2, TRAF6, TAK1, CARD9, CARD10 (or CARMA3), CARD11 (or CARMA1), CARD14 (or CARMA2), FABP5, TAB1, TAB2, TAB3, TAK1, IKKα, IKKβ, IKKγ, AP11, AP12, AP13, AP14 or A20.

9-10. (canceled)

11. The method of claim 1, wherein the FABP5 inhibitor binds irreversibly to FABP5 to form a covalent bond.

12. The method of claim 1, wherein the FABP5 inhibitor is a compound of formula (I) or a pharmaceutically acceptable salt or a stereoisomer thereof: 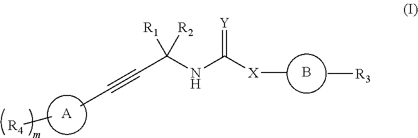
wherein,
A represents aryl or heteroaryl;
X represents N—R, or is absent;
Y represents O, S or NCN;
B represents aryl, cycloalkyl or heterocycloalkyl; wherein the aryl, cycloalkyl or heterocycloalkyl are optionally substituted with one or more of an alkyl group, a halogen or an oxo group;
R₁ represents alkyl; R₂ represents hydrogen or alkyl; or R₁ and R₂ together with the carbon atoms to which they are attached form a 3- to 5-membered cycloalkyl ring;
R₃ represents —C(O)Rₐ, —S(O)₂Rₐ, —NHS(O)₂R₁, —NR_{b}C(O)Rₐ, ═NORₐ, heteroaryl, heterocycloalkyl or (heterocycloalkyl)alkyl-; wherein the heteroaryl and heterocycloalkyl are optionally substituted with one or more of an alkyl group, a halogen, an oxo group or —C(O)Rₓ;
R₄ represents alkyl, halo, haloalkyl, cyano, alkoxy, aryloxy, alkoxyaryl, hydroxyalkyl, acetylene, acyl, hydroxy, cycloalkyl or —N(Rₓ)₂; wherein the cycloalkyl is optionally substituted with alkyl;
Rₐ represents alkyl, alkenyl, haloalkyl, cycloalkyl or heterocycloalkyl; wherein the alkyl, alkenyl, haloalkyl, cycloalkyl and heterocycloalkyl are optionally substituted with one or more groups that are alkyl, halogen, aryl, cycloalkyl, haloalkyl, amino, amido, alkylamino, aminoalkyl, hydroxyl, cyano, alkoxy, alkoxyaryl, aryloxy, hydroxyalkyl, carboxylic acid, ester, thioester, oxo(═O) or —C(O)Rₓ;
Rₓ represents hydrogen, alkyl, alkenyl, acyl or —C(O)-cycloalkyl;
R_{y} represents hydrogen or alkyl;
R_{b} represents hydrogen, alkyl or alkenyl; and
m represents 0, 1, 2 or 3.

13. (canceled)

14. The method of claim 12, wherein B represents

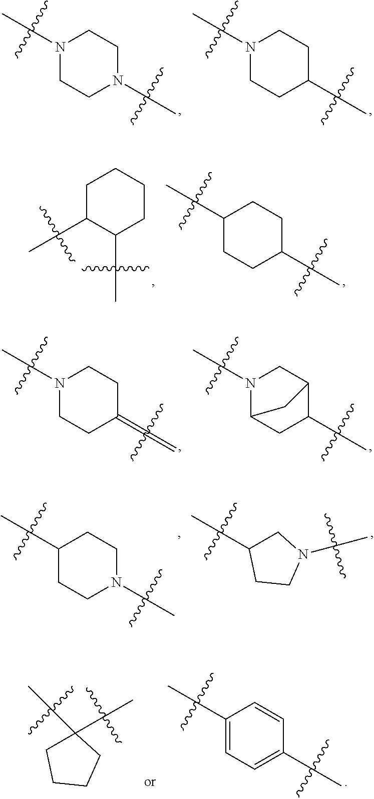

15. The method of claim 12, wherein R₁ represents alkyl; and R₂ represents hydrogen; or R₁ and R₂ together with the carbon atoms to which they are attached form a cyclopropyl ring or a cyclopentyl ring.

16-17. (canceled)

18. The method of claim 1, wherein the FABP5 inhibitor has a structure of compound of formula (IA):

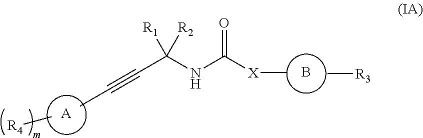

19-22. (canceled)

23. The method of claim 8, wherein the FABP5 inhibitor has a structure of compound of formula (IB):

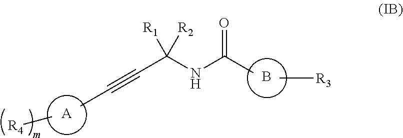

24-25. (canceled)

26. The method of claim 8, wherein the FABP5 inhibitor has a structure of compound of formula (IC), formula (ID) or formula (IE):

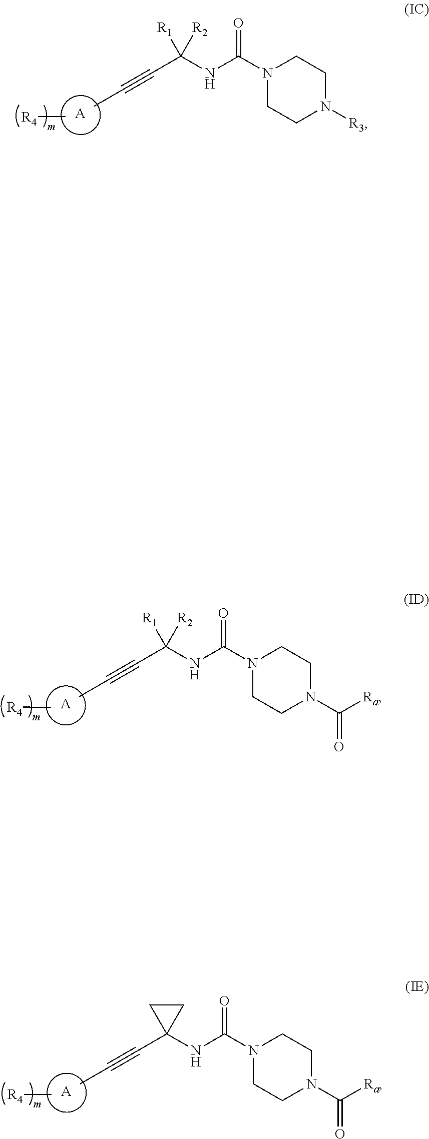

27-34. (canceled)

35. The method of claim 8, wherein the FABP5 inhibitor has a structure of compound of formula (IF), (IG) or (IH):

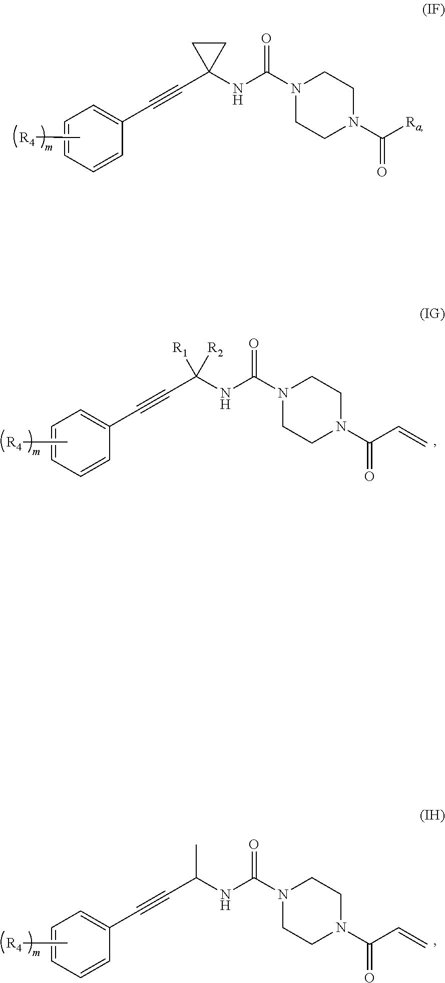

36. The method of claim 1, wherein the FABP5 inhibitor is a compound that is:

| Compound | Structure |
|---|---|
| 1 | 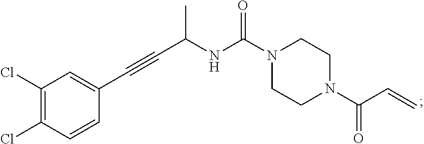 [SMILES: C=CC(=O)N1CCN(C(=O)NC(C)C#Cc2ccc(Cl)c(Cl)c2)CC1] |
| 1a | 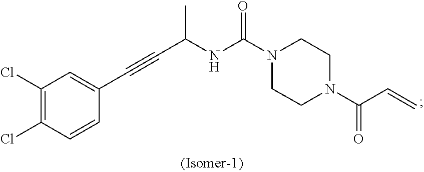 [SMILES: C=CC(=O)N1CCN(C(=O)NC(C)C#Cc2ccc(Cl)c(Cl)c2)CC1] |
| 1b | 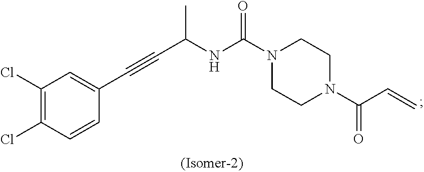 [SMILES: C=CC(=O)N1CCN(C(=O)NC(C)C#Cc2ccc(Cl)c(Cl)c2)CC1] |
| 2 | 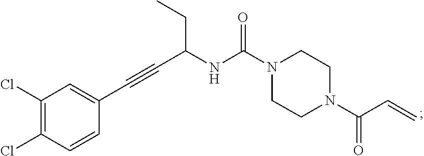 [SMILES: C=CC(=O)N1CCN(C(=O)NC(C#Cc2ccc(Cl)c(Cl)c2)CC)CC1] |
| 2a | 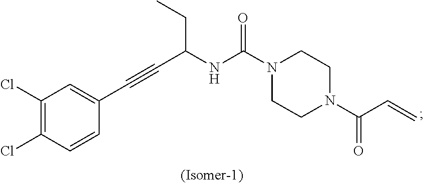 [SMILES: C=CC(=O)N1CCN(C(=O)NC(C#Cc2ccc(Cl)c(Cl)c2)CC)CC1] |
| 2b | 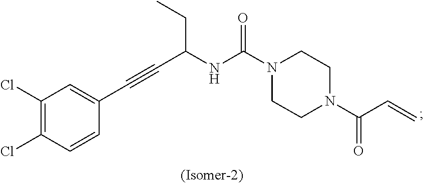 [SMILES: C=CC(=O)N1CCN(C(=O)NC(C#Cc2ccc(Cl)c(Cl)c2)CC)CC1] |
| 3 | 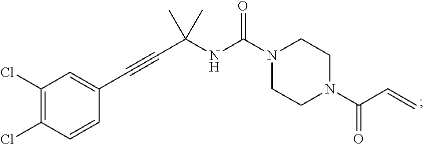 [SMILES: C=CC(=O)N1CCN(C(=O)NC(C)(C)C#Cc2ccc(Cl)c(Cl)c2)CC1] |
| 4 | 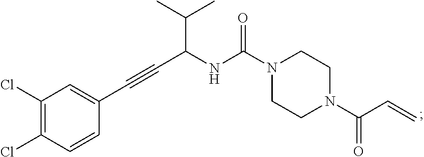 [SMILES: C=CC(=O)N1CCN(C(=O)NC(C#Cc2ccc(Cl)c(Cl)c2)C(C)C)CC1] |
| 5 | 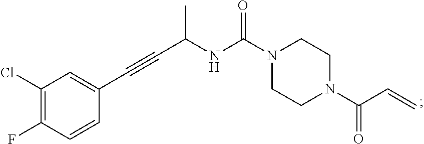 [SMILES: C=CC(=O)N1CCN(C(=O)NC(C)C#Cc2ccc(F)c(Cl)c2)CC1] |
| 6 | 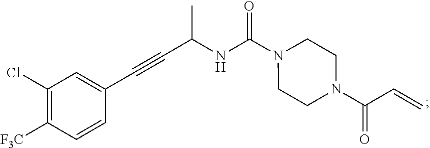 [SMILES: C=CC(=O)N1CCN(C(=O)NC(C)C#Cc2ccc(C(F)(F)F)c(Cl)c2)CC1] |
| 7 | 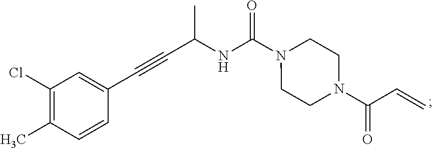 [SMILES: C=CC(=O)N1CCN(C(=O)NC(C)C#Cc2ccc(C)c(Cl)c2)CC1] |
| 8 | 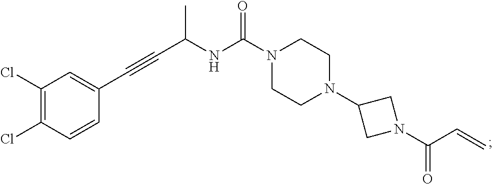 [SMILES: C=CC(=O)N1CC(N2CCN(C(=O)NC(C)C#Cc3ccc(Cl)c(Cl)c3)CC2)C1] |
| 9 | 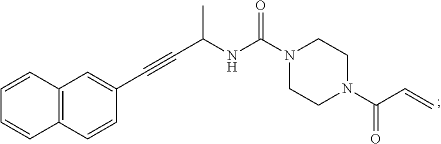 [SMILES: C=CC(=O)N1CCN(C(=O)NC(C)C#Cc2ccc3ccccc3c2)CC1] |
| 10 | 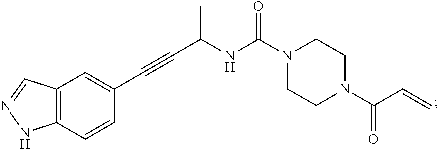 [SMILES: C=CC(=O)N1CCN(C(=O)NC(C)C#Cc2ccc3[nH]ncc3c2)CC1] |
| 11 | 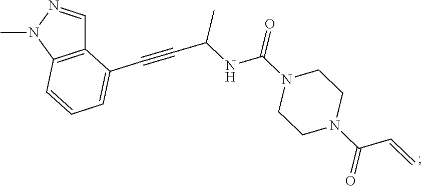 [SMILES: C=CC(=O)N1CCN(C(=O)NC(C)C#Cc2cccc3c2cnn3C)CC1] |
| 12 | 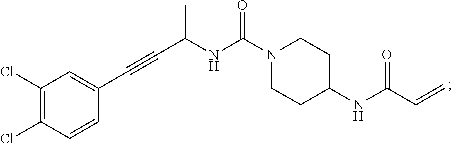 [SMILES: C=CC(=O)NC1CCN(C(=O)NC(C)C#Cc2ccc(Cl)c(Cl)c2)CC1] |
| 13 | 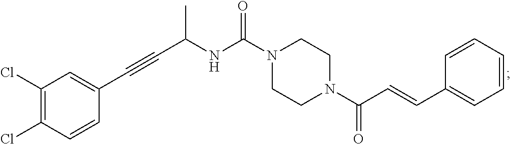 [SMILES: CC(C#Cc1ccc(Cl)c(Cl)c1)NC(=O)N1CCN(C(=O)/C=C/c2ccccc2)CC1] |
| 14 | 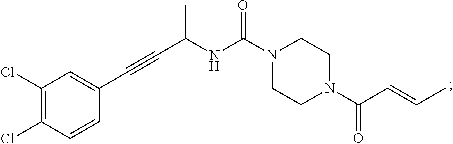 [SMILES: C/C=C/C(=O)N1CCN(C(=O)NC(C)C#Cc2ccc(Cl)c(Cl)c2)CC1] |
| 15 | 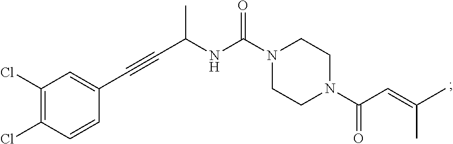 [SMILES: CC(C)=CC(=O)N1CCN(C(=O)NC(C)C#Cc2ccc(Cl)c(Cl)c2)CC1] |
| 16 | 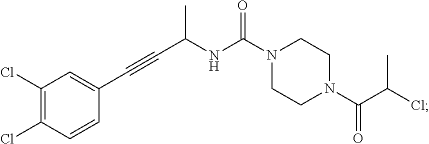 [SMILES: CC(C#Cc1ccc(Cl)c(Cl)c1)NC(=O)N1CCN(C(=O)C(C)Cl)CC1] |
| 17 | 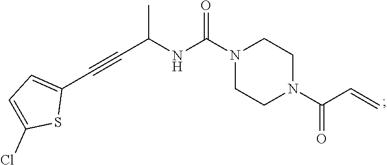 [SMILES: C=CC(=O)N1CCN(C(=O)NC(C)C#Cc2ccc(Cl)s2)CC1] |
| 18 | 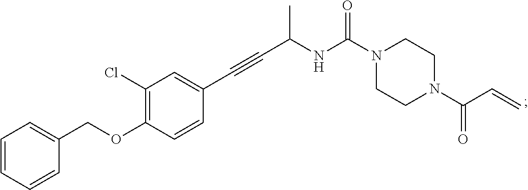 [SMILES: C=CC(=O)N1CCN(C(=O)NC(C)C#Cc2ccc(OCc3ccccc3)c(Cl)c2)CC1] |
| 19 | 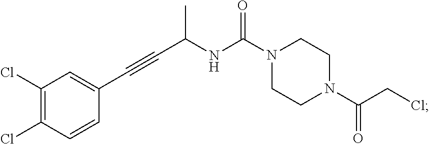 [SMILES: CC(C#Cc1ccc(Cl)c(Cl)c1)NC(=O)N1CCN(C(=O)CCl)CC1] |
| 20 | 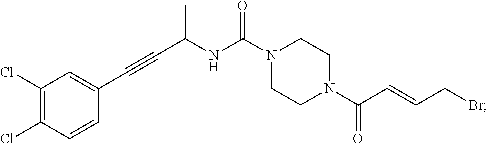 [SMILES: CC(C#Cc1ccc(Cl)c(Cl)c1)NC(=O)N1CCN(C(=O)/C=C/CBr)CC1] |
| 21 | 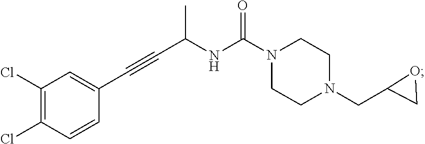 [SMILES: CC(C#Cc1ccc(Cl)c(Cl)c1)NC(=O)N1CCN(CC2CO2)CC1] |
| 22 | 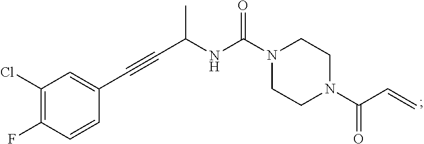 [SMILES: C=CC(=O)N1CCN(C(=O)NC(C)C#Cc2ccc(F)c(Cl)c2)CC1] |
| 23 | 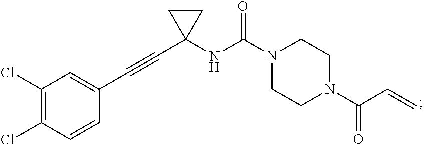 [SMILES: C=CC(=O)N1CCN(C(=O)NC2(C#Cc3ccc(Cl)c(Cl)c3)CC2)CC1] |
| 24 | 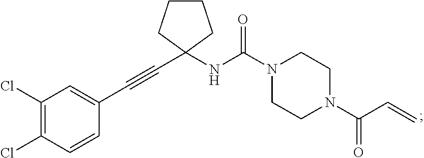 [SMILES: C=CC(=O)N1CCN(C(=O)NC2(C#Cc3ccc(Cl)c(Cl)c3)CCCC2)CC1] |
| 25 | 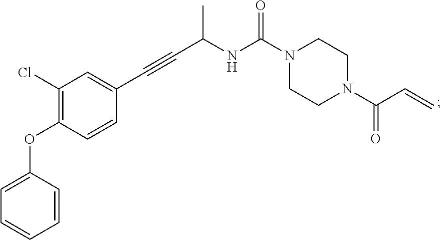 [SMILES: C=CC(=O)N1CCN(C(=O)NC(C)C#Cc2ccc(Oc3ccccc3)c(Cl)c2)CC1] |
| 26 | 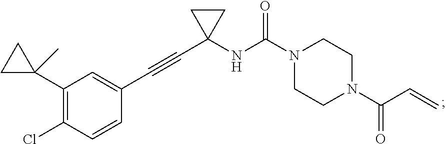 [SMILES: C=CC(=O)N1CCN(C(=O)NC2(C#Cc3ccc(Cl)c(C4(C)CC4)c3)CC2)CC1] |
| 27 | 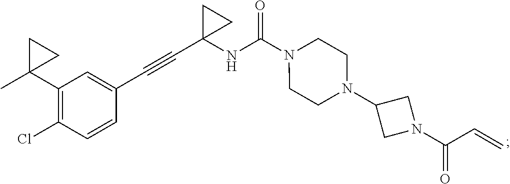 [SMILES: C=CC(=O)N1CC(N2CCN(C(=O)NC3(C#Cc4ccc(Cl)c(C5(C)CC5)c4)CC3)CC2)C1] |
| 28 | 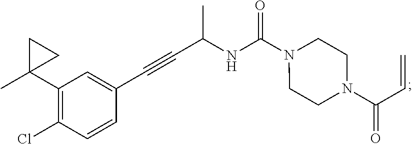 [SMILES: C=CC(=O)N1CCN(C(=O)NC(C)C#Cc2ccc(Cl)c(C3(C)CC3)c2)CC1] |
| 29 | 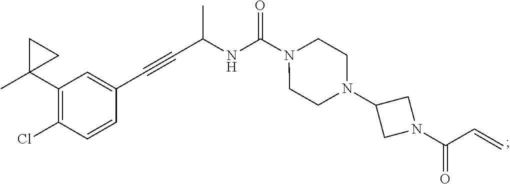 [SMILES: C=CC(=O)N1CC(N2CCN(C(=O)NC(C)C#Cc3ccc(Cl)c(C4(C)CC4)c3)CC2)C1] |
| 30 | 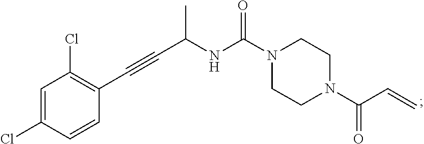 [SMILES: C=CC(=O)N1CCN(C(=O)NC(C)C#Cc2ccc(Cl)cc2Cl)CC1] |
| 31 | 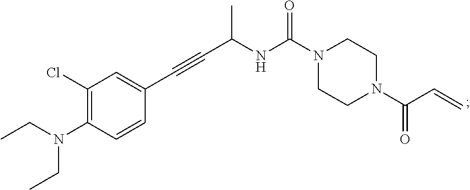 [SMILES: C=CC(=O)N1CCN(C(=O)NC(C)C#Cc2ccc(N(CC)CC)c(Cl)c2)CC1] |
| 32 | 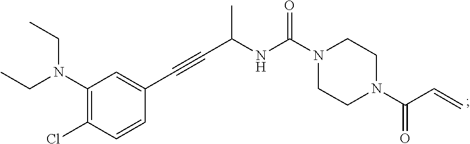 [SMILES: C=CC(=O)N1CCN(C(=O)NC(C)C#Cc2ccc(Cl)c(N(CC)CC)c2)CC1] |
| 33 | 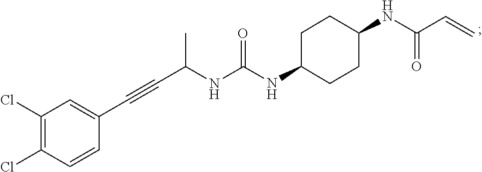 [SMILES: C=CC(=O)N[C@H]1CC[C@@H](NC(=O)NC(C)C#Cc2ccc(Cl)c(Cl)c2)CC1] |
| 34 | 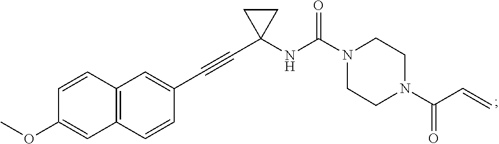 [SMILES: C=CC(=O)N1CCN(C(=O)NC2(C#Cc3ccc4cc(OC)ccc4c3)CC2)CC1] |
| 35 | 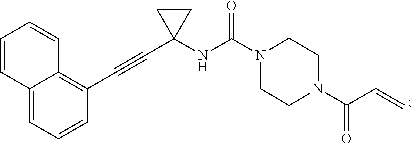 [SMILES: C=CC(=O)N1CCN(C(=O)NC2(C#Cc3cccc4ccccc34)CC2)CC1] |
| 36 | 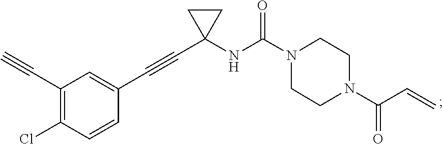 [SMILES: C#Cc1cc(C#CC2(NC(=O)N3CCN(C(=O)C=C)CC3)CC2)ccc1Cl] |
| 37 | 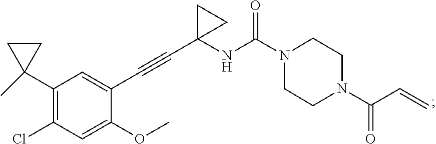 [SMILES: C=CC(=O)N1CCN(C(=O)NC2(C#Cc3cc(C4(C)CC4)c(Cl)cc3OC)CC2)CC1] |
| 38 | 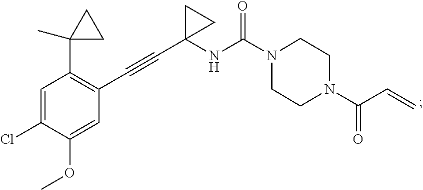 [SMILES: C=CC(=O)N1CCN(C(=O)NC2(C#Cc3cc(OC)c(Cl)cc3C3(C)CC3)CC2)CC1] |
| 39 | 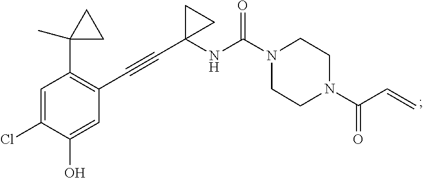 [SMILES: C=CC(=O)N1CCN(C(=O)NC2(C#Cc3cc(O)c(Cl)cc3C3(C)CC3)CC2)CC1] |
| 40 | 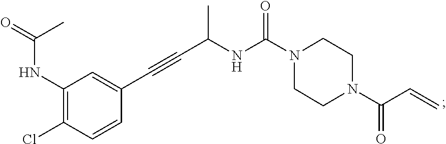 [SMILES: C=CC(=O)N1CCN(C(=O)NC(C)C#Cc2ccc(Cl)c(NC(C)=O)c2)CC1] |
| 41 | 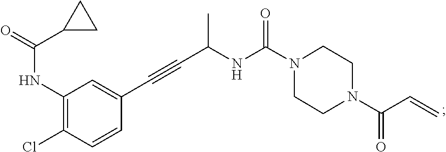 [SMILES: C=CC(=O)N1CCN(C(=O)NC(C)C#Cc2ccc(Cl)c(NC(=O)C3CC3)c2)CC1] |
| 42 | 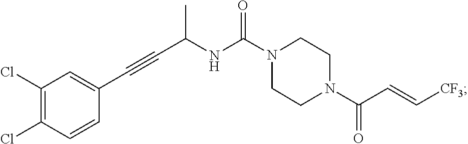 [SMILES: CC(C#Cc1ccc(Cl)c(Cl)c1)NC(=O)N1CCN(C(=O)/C=C/C(F)(F)F)CC1] |
| 42a | 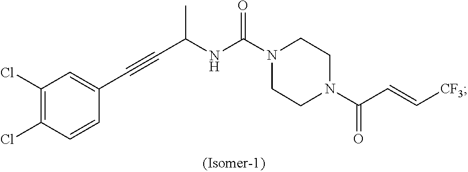 [SMILES: CC(C#Cc1ccc(Cl)c(Cl)c1)NC(=O)N1CCN(C(=O)/C=C/C(F)(F)F)CC1] |
| 42b | 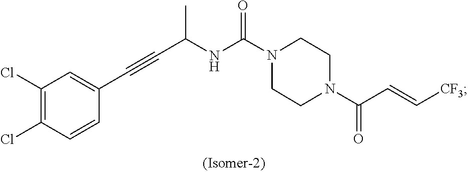 [SMILES: CC(C#Cc1ccc(Cl)c(Cl)c1)NC(=O)N1CCN(C(=O)/C=C/C(F)(F)F)CC1] |
| 43 | 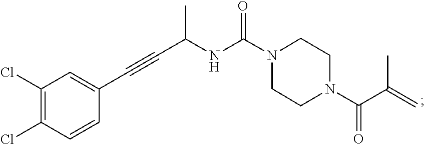 [SMILES: C=C(C)C(=O)N1CCN(C(=O)NC(C)C#Cc2ccc(Cl)c(Cl)c2)CC1] |
| 44 | 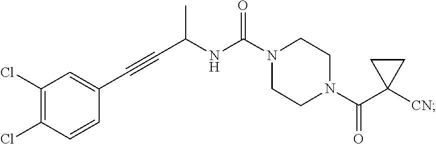 [SMILES: CC(C#Cc1ccc(Cl)c(Cl)c1)NC(=O)N1CCN(C(=O)C2(C#N)CC2)CC1] |
| 45 | 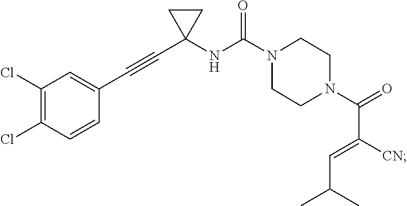 [SMILES: CC(C)/C=C(\C#N)C(=O)N1CCN(C(=O)NC2(C#Cc3ccc(Cl)c(Cl)c3)CC2)CC1] |
| 46 | 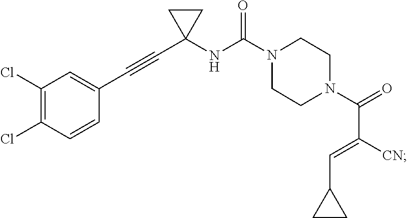 [SMILES: N#C/C(=C\C1CC1)C(=O)N1CCN(C(=O)NC2(C#Cc3ccc(Cl)c(Cl)c3)CC2)CC1] |
| 47 | 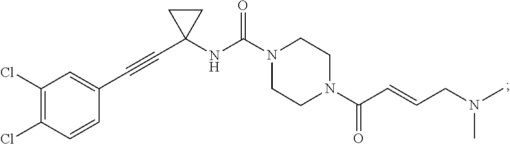 [SMILES: CN(C)C/C=C/C(=O)N1CCN(C(=O)NC2(C#Cc3ccc(Cl)c(Cl)c3)CC2)CC1] |
| 48a | 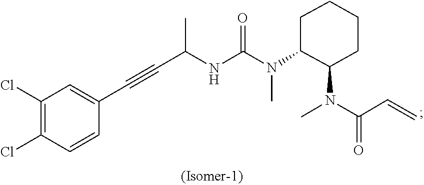 [SMILES: C=CC(=O)N(C)[C@@H]1CCCC[C@H]1N(C)C(=O)NC(C)C#Cc1ccc(Cl)c(Cl)c1] |
| 48b | 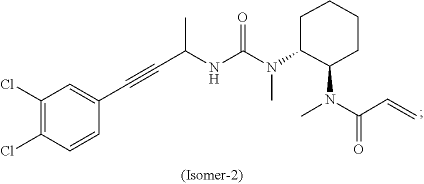 [SMILES: C=CC(=O)N(C)[C@@H]1CCCC[C@H]1N(C)C(=O)NC(C)C#Cc1ccc(Cl)c(Cl)c1] |
| 49 | 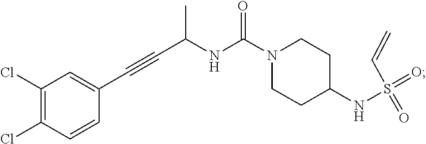 [SMILES: C=CS(=O)(=O)NC1CCN(C(=O)NC(C)C#Cc2ccc(Cl)c(Cl)c2)CC1] |
| 50 | 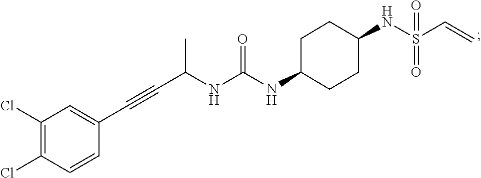 [SMILES: C=CS(=O)(=O)N[C@H]1CC[C@@H](NC(=O)NC(C)C#Cc2ccc(Cl)c(Cl)c2)CC1] |
| 51 | 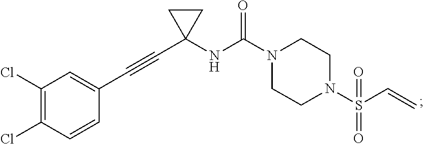 [SMILES: C=CS(=O)(=O)N1CCN(C(=O)NC2(C#Cc3ccc(Cl)c(Cl)c3)CC2)CC1] |
| 52 | 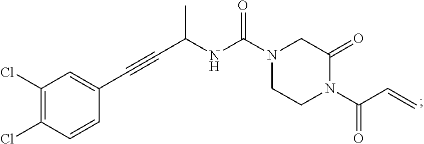 [SMILES: C=CC(=O)N1CCN(C(=O)NC(C)C#Cc2ccc(Cl)c(Cl)c2)CC1=O] |
| 53 | 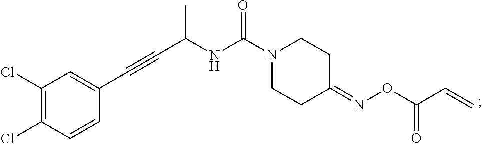 [SMILES: C=CC(=O)ON=C1CCN(C(=O)NC(C)C#Cc2ccc(Cl)c(Cl)c2)CC1] |
| 54 | 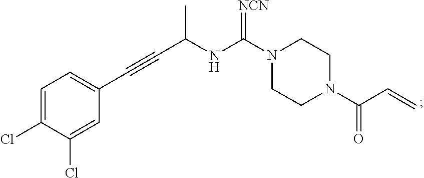 |
| 55 | 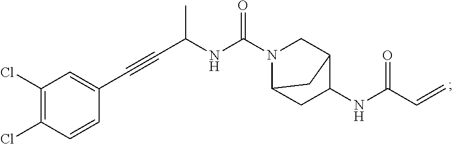 [SMILES: C=CC(=O)NC1CC2CC1CN2C(=O)NC(C)C#Cc1ccc(Cl)c(Cl)c1] |
| 56 | 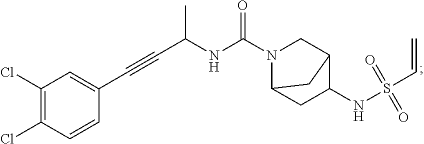 [SMILES: C=CS(=O)(=O)NC1CC2CC1CN2C(=O)NC(C)C#Cc1ccc(Cl)c(Cl)c1] |
| 57 | 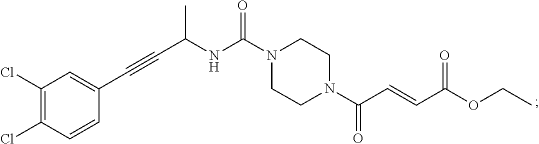 [SMILES: CCOC(=O)/C=C/C(=O)N1CCN(C(=O)NC(C)C#Cc2ccc(Cl)c(Cl)c2)CC1] |
| 58 | 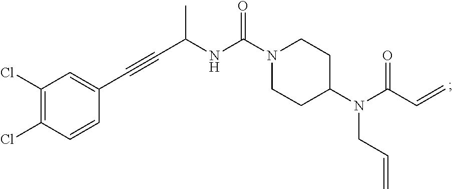 [SMILES: C=CCN(C(=O)C=C)C1CCN(C(=O)NC(C)C#Cc2ccc(Cl)c(Cl)c2)CC1] |
| 59 | 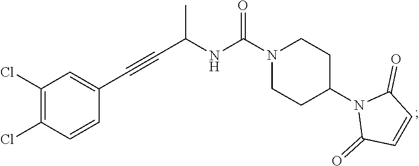 [SMILES: CC(C#Cc1ccc(Cl)c(Cl)c1)NC(=O)N1CCC(N2C(=O)C=CC2=O)CC1] |
| 60 | 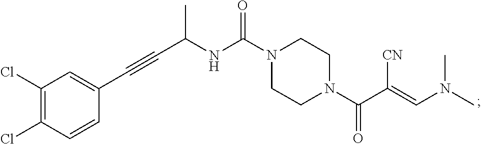 [SMILES: CC(C#Cc1ccc(Cl)c(Cl)c1)NC(=O)N1CCN(C(=O)/C(C#N)=C/N(C)C)CC1] |
| 61 | 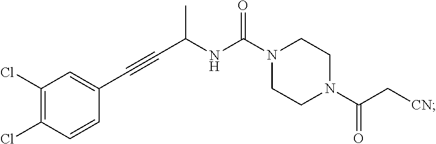 [SMILES: CC(C#Cc1ccc(Cl)c(Cl)c1)NC(=O)N1CCN(C(=O)CC#N)CC1] |
| 62 | 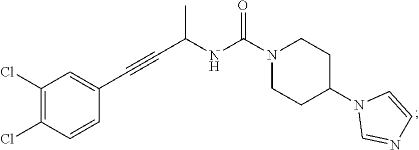 [SMILES: CC(C#Cc1ccc(Cl)c(Cl)c1)NC(=O)N1CCC(n2ccnc2)CC1] |
| 63 | 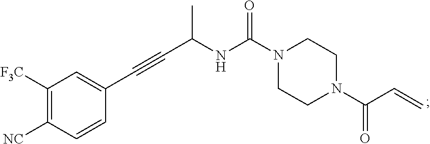 [SMILES: C=CC(=O)N1CCN(C(=O)NC(C)C#Cc2ccc(C#N)c(C(F)(F)F)c2)CC1] |
| 64 | 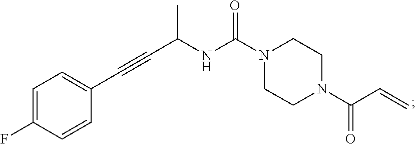 [SMILES: C=CC(=O)N1CCN(C(=O)NC(C)C#Cc2ccc(F)cc2)CC1] |
| 65 | 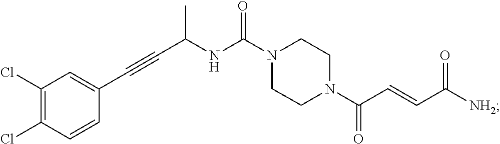 [SMILES: CC(C#Cc1ccc(Cl)c(Cl)c1)NC(=O)N1CCN(C(=O)/C=C/C(N)=O)CC1] |
| 66 | 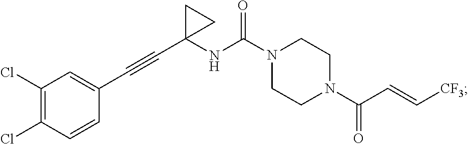 [SMILES: O=C(/C=C/C(F)(F)F)N1CCN(C(=O)NC2(C#Cc3ccc(Cl)c(Cl)c3)CC2)CC1] |
| 67 | 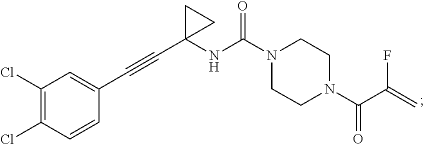 [SMILES: C=C(F)C(=O)N1CCN(C(=O)NC2(C#Cc3ccc(Cl)c(Cl)c3)CC2)CC1] |
| 68 | 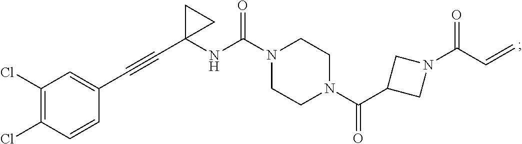 [SMILES: C=CC(=O)N1CC(C(=O)N2CCN(C(=O)NC3(C#Cc4ccc(Cl)c(Cl)c4)CC3)CC2)C1] |
| 69 | 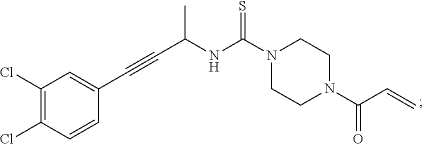 [SMILES: C=CC(=O)N1CCN(C(=S)NC(C)C#Cc2ccc(Cl)c(Cl)c2)CC1] |
| 70 | 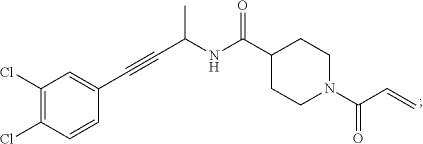 [SMILES: C=CC(=O)N1CCC(C(=O)NC(C)C#Cc2ccc(Cl)c(Cl)c2)CC1] |
| 71 | 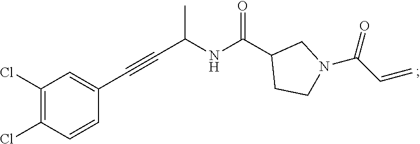 [SMILES: C=CC(=O)N1CCC(C(=O)NC(C)C#Cc2ccc(Cl)c(Cl)c2)C1] |
| 72 | 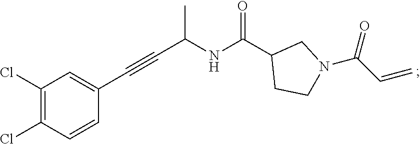 [SMILES: C=CC(=O)N1CCC(C(=O)NC(C)C#Cc2ccc(Cl)c(Cl)c2)C1] |
| 73 | 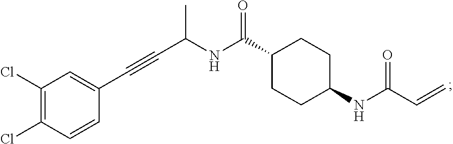 [SMILES: C=CC(=O)N[C@H]1CC[C@H](C(=O)NC(C)C#Cc2ccc(Cl)c(Cl)c2)CC1] |
| 74 | 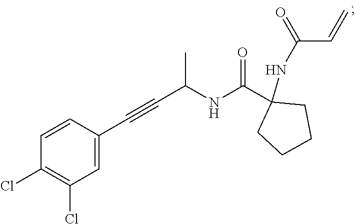 [SMILES: C=CC(=O)NC1(C(=O)NC(C)C#Cc2ccc(Cl)c(Cl)c2)CCCC1] |
| 75 | 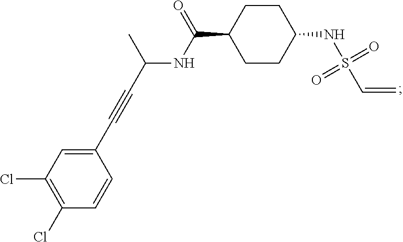 [SMILES: C=CS(=O)(=O)N[C@H]1CC[C@H](C(=O)NC(C)C#Cc2ccc(Cl)c(Cl)c2)CC1] |
| 76 | 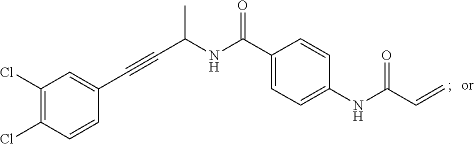 [SMILES: C=CC(=O)Nc1ccc(C(=O)NC(C)C#Cc2ccc(Cl)c(Cl)c2)cc1] |
| 77 | 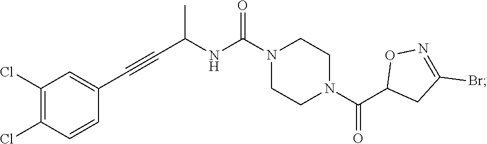 [SMILES: CC(C#Cc1ccc(Cl)c(Cl)c1)NC(=O)N1CCN(C(=O)C2CC(Br)=NO2)CC1] |

or a pharmaceutically acceptable salt or a stereoisomer thereof.

37. The method of claim 1, wherein the step of contacting the cancer cell occurs in a subject in need thereof, thereby treating a cancer associated with the deregulated lymphocyte receptor signaling pathway.

38. The method of claim 37, wherein the cancer is characterized by aberrant activity of a B-cell receptor signalling pathway or a T-cell receptor signaling pathway.

39-40. (canceled)

41. A method of treating a cancer having associated therewith a deregulated lymphocyte receptor signaling pathway in a subject in need thereof comprising administering to the subject a therapeutically effective amount of a FABP5 inhibitor; wherein the cancer is a B-cell cancer or a T-cell cancer and wherein treatment thereof comprises inhibiting growth of B-cell tumor cell, growth of T-cell tumor cells or metastasis thereof or a combination thereof.

42-43. (canceled)

44. The method of claim 37, wherein the cancer is a B-cell cancer comprising chronic lymphocytic leukemia (CLL), small lymphocytic lymphoma (SLL), diffuse large B-cell lymphoma (DLBCL), activated B-cell diffuse large B-cell lymphoma (ABC-DLBCL), germinal center diffuse large B-cell lymphoma (GCB DLBCL), primary mediastinal B-cell lymphoma (PMBL), non-Hodgkin lymphoma, Burkitt's lymphoma, follicular lymphoma, immunoblastic large cell lymphoma, precursor B-lymphoblastic lymphoma, precursor B-cell acute lymphoblastic leukemia, hairy cell leukemia, mantle cell lymphoma, B cell prolymphocytic leukaemia, lymphoplasmacytic lymphoma/Waldenstrom macroglobulinemia, splenic marginal zone lymphoma, plasma cell myeloma, plasmacytoma, extranodal marginal zone B cell lymphoma, nodal marginal zone B cell lymphoma, mediastinal (thymic) large B cell lymphoma, intravascular large B cell lymphoma, primary effusion lymphoma, or lymphomatoid granulomatosis.

45-47. (canceled)

48. The method of claim 37, wherein the cancer is a T-cell cancer that is a T-cell malignancy comprising peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or a treatment-related T-cell lymphoma.

49-50. (canceled)

52. A method of treating a solid tumor in a subject in need thereof comprising administering to the subject a therapeutically effective amount of a FABP5 inhibitor; wherein the solid tumor is a tumor of the prostate, brain, head and neck, cervix, colon, pancreas, bladder, gastric, skin, esophagus, liver, bile duct or kidney.

53. (canceled)

54. The method of claim 41, wherein the FABP5 inhibitor is a compound of formula (I) or a pharmaceutically acceptable salt or a stereoisomer thereof: 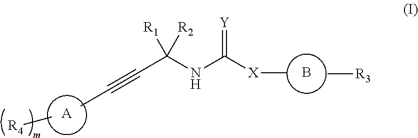
wherein,
A represents aryl or heteroaryl;
X represents N—R, or is absent;
Y represents O, S or NCN;
B represents aryl, cycloalkyl or heterocycloalkyl; wherein the aryl, cycloalkyl or heterocycloalkyl are optionally substituted with one or more of an alkyl group, a halogen or an oxo group;
R₁ represents alkyl; R₂ represents hydrogen or alkyl; or R₁ and R₂ together with the carbon atoms to which they are attached form a 3- to 5-membered cycloalkyl ring;
R₃ represents —C(O)Rₐ, —S(O)₂Rₐ, —NHS(O)₂Rₐ, —NR_{b}C(O)R_{a,} ═NORₐ, heteroaryl, heterocycloalkyl or (heterocycloalkyl)alkyl-; wherein the heteroaryl and heterocycloalkyl are optionally substituted with one or more of an alkyl group, a halogen, an oxo group or —C(O)Rₓ;
R₄ represents alkyl, halo, haloalkyl, cyano, alkoxy, aryloxy, alkoxyaryl, hydroxyalkyl, acetylene, acyl, hydroxy, cycloalkyl or —N(Rₓ)₂; wherein the cycloalkyl is optionally substituted with alkyl;
Rₐ represents alkyl, alkenyl, haloalkyl, cycloalkyl or heterocycloalkyl; wherein the alkyl, alkenyl, haloalkyl, cycloalkyl and heterocycloalkyl are optionally substituted with one or more groups that are alkyl, halogen, aryl, cycloalkyl, haloalkyl, amino, amido, alkylamino, aminoalkyl, hydroxyl, cyano, alkoxy, alkoxyaryl, aryloxy, hydroxyalkyl, carboxylic acid, ester, thioester, oxo(═O) or —C(O)Rₓ;
Rₓ represents hydrogen, alkyl, alkenyl, acyl or —C(O)-cycloalkyl;
R_{y} represents hydrogen or alkyl;
R_{b} represents hydrogen, alkyl or alkenyl; and
m represents 0, 1, 2 or 3.

55-57. (canceled)
